(19) United States
(12) Patent Application Publication
(10) Pub. No.: US 2021/0031060 A1
(43) Pub. Date: Feb. 4, 2021

(54) METHODS, SYSTEMS AND APPARATUSES FOR SUPPLYING BREATHABALE GASES

(71) Applicant: VLADIMIR BELYAEV, GREER, SC (US)

(72) Inventor: VLADIMIR BELYAEV, GREER, SC (US)

(21) Appl. No.: 17/041,386

(22) PCT Filed: Mar. 25, 2019
(86) PCT No.: PCT/US19/23948

§ 371 Date: Sep. 24, 2020

Related U.S. Application Data
(60) Provisional application No. 62/647,719, filed on Mar. 24, 2018.

(51) Int. Cl. A62B 7/02; A62B 31/00; A62B 7/06; A62B 15/00; G06Q 20/10; G07F 15/00; F17C 3/00

(52) U.S. Cl. CPC A62B 7/02; F17C 2223/0161; A62B 7/06; A62B 15/00; G06Q 20/10; G07F 15/001; F17C 3/00; F17C 2270/025; F17C 2223/0123; F17C 2201/058; F17C 2227/0157; F17C 2201/0109; F17C 2201/054; F17C 2205/0335; F17C 2221/011; F17C 2221/014; A62B 31/00

(57) ABSTRACT

A strong unsolved need exists to provide people with breathable gases for inhaling instead of ambient air, when people live in areas, where the ambient air is polluted with toxic gases and particulate matters. The present invention solves this need by disclosing a breathing apparatus, a dispenser of breathable gases, and a distributor of breathable gases, for supplying breathable gases to people, exposed to polluted and contaminated outdoor and indoor ambient air.

### TECHNICAL FIELD

The present invention relates to methods, systems and apparatuses for supplying breathable gases to civilian people, exposed to polluted and contaminated ambient air.

A strong unsolved need exists to provide people with breathable gases for inhaling instead of ambient air, when people live in areas, where the ambient air is polluted with toxic gases and particulate matters.

Such areas can be found, for example, nearby roads and highways, where vehicles utilize hydrocarbon fuels and bio fuels. Exhaust gases of these vehicles comprise pollutants and contaminants, like nitrogen oxides, carbon monoxide, particulate matters etc. Physical wear out of tires and road surfaces leads to increased particulate matters concentration in ambient air.

Relatively high concentrations of particulate matters can be found in underground atmosphere, due to wear out of elements of escalators and wagons, as well as due to lack of ventilation.

Another example is areas in close proximity to industrial objects, like manufacturing plants, chemical plants and power plants. Exhaust gases of these objects comprise pollutants and contaminants.

Worthy to note that certain levels of concentrations of pollutants and contaminants may not be immediately dangerous or hazardous. However long term exposure to such levels of concentrations of pollutants and contaminants in ambient air, especially while inhaling it, may lead to some diseases.

In many cases ambient air may be simply not pleasant for breathing, for example, nearby laundries, kitchens, smoking areas, etc.

Indoor materials and surfaces pollution can significantly reduce quality of inhaled air.

Present invention provides breathable gases for supplemental inhaling to people exposed to polluted and contaminated ambient outdoor and indoor air.

### BACKGROUND ART

Self-contained breathing apparatuses supply breathable gases to firefighters, industrial workers, miners, rescue teams, aviation pilots, and astronauts. Self-contained underwater breathing apparatuses supply breathable gases to divers.

Compressed air refill stations refill self-contained breathing apparatuses and self-contained underwater breathing apparatuses.

Medical breathing apparatuses provide patients with high quality breathing gases, cleaned from biological contaminants, like bacterium, spores, and other harmful microorganisms.

Medical breathing apparatuses control composition of inhaling gases. For example, oxygen enriching devices exist, for this purpose.

In home use oxygen refill stations refill medical breathing devices, enriching inhaled ambient air with oxygen.

Continuous positive airway pressure devices are used by people who have breathing problems to keep the airways open.

Ventilation systems use filtration and absorption techniques to clean up the ambient air, taken from immediate proximity to the location of use, for example, outdoor atmosphere, prior to supply this air to indoor spaces for inhaling by residents.

Bubble apparatuses create micro-climate within a bubble, made of relatively impermeable materials, relatively isolate certain space from ambient air and maintain a predefined conditions inside the bubble, such as chemical composition, pressure, etc.

### DISCLOSURE OF INVENTION

The invention is disclosed in a level of details, that would allow one skilled in the art to conceive the invention and become able to develop relevant products applying engineering skills and knowledge of relevant techniques, design procedures, and technical standards, and only essential elements of methods, systems and apparatuses are described in the disclosure. Some elements, such as pumps, valves, compressors, casings, enclosures, frames, mountings, joints, couplings, communications, pipelines, manifolds, ducts, receivers, etc. are considered as regular engineering elements and omitted therefore.

FIG. 1A illustrates a system configured to supply breathable gases to civilian people. The system comprises a source of breathable gases 10M1, a breathable gases dispensing system 100M1 in communication with the source of breathable gases 10M1 and in temporary communication with a breathing system 200M1. The source of breathable gases 10M1 generates, or stores, or generates and stores, and conducts the breathable gases to the breathable gases dispensing system 100M1. The breathable gases dispensing system 100M1 receives the breathable gases from the source of breathable gases 10M1 and dispenses the breathable gases to the breathing system 200M1. In more detail, the breathable gases dispensing system 100M1 comprises a dispenser of breathable gases 101 in communication with the source of breathable gases 10M1 and a communication unit 102 in communication with the dispenser of breathable gases 101. In more detail, the breathing system 200M1 comprises a container of breathable gases 201 in a temporary communication with the dispenser of breathable gases 101 and an inhalation unit 220 in communication with the container of breathable gases 201. The communication unit 102 receives an input from a user preferably, but not limited to, in a form of a payment transaction, the input defines an amount of breathable gases to be dispensed by the dispenser of breathable gases 101 to the container of breathable gases 201. The communication unit 102 converts the input from a user to a signal to the dispenser of breathable gases 101 to dispense the predefined amount of breathable gases to the container of breathable gases 201. The dispenser of breathable gases 101 receives breathable gases from the source of breathable gases 10M1, receives the signal from the communication unit 102 to dispense the predefined amount of breathable gases to the container of breathable gases 201, and dispenses the predefined amount of breathable gases to the container of breathable gases 201 in response to the signal from the communication unit 102. The dispenser of breathable gases 101 communicates data of interest, such as the amount of dispensed breathable gases and a pressure of the breathable gases, to the communication unit 102 to be available for a current user. The data of interest can additionally be communicated to a smartphone or a tablet PC of the user. When the predefined amount of breathable gases is dispensed to the container of breathable gases 201, the connection between the dispenser of breathable gases 101 and the container of breathable gases 201 should be disconnected, and another container of breathable gases 201 can be connected to the dispenser of breathable gases 101 for dispensing breathable gases. The container of breathable gases 201, containing dispensed breathable gases, when disconnected from the dispenser of breathable gases 101, becomes a portable source of breathable gases. The container of breathable gases 201 conducts the breathable gases to the inhalation unit 220. The inhalation unit 220 receives the breathable gases from the container of breathable gases 201 and releases the breathable gases to inhalation organs of a user for a period of time corresponding to the capacity of the container of breathable gases 201.

FIG. 1B illustrates another arrangement of a system configured to supply breathable gases to civilian people. The system comprises a source of breathable gases 10M1, a breathable gases distributing system 100M2 in communication with the source of breathable gases 10M1 and in temporary communication with a breathing system 200M2. The source of breathable gases 10M1 generates, or stores, or generates and stores, and conducts the breathable gases to the breathable gases distributing system 100M2. The breathable gases distributing system 100M2 receives the breathable gases from the source of breathable gases 10M1 and conducts the breathable gases to the breathing system 200M2. In more detail, the breathable gases distributing system 100M2 comprises a distributor of breathable gases 103 in communication with the source of breathable gases 10M1 and a communication unit 102 in communication with the distributor of breathable gases 103. In more detail, the breathing system 200M1 comprises an inhalation unit 220 in communication with the distributor of breathable gases 103. The communication unit 102 receives an input from a user preferably, but not limited to, in a form of a payment transaction, the input defines a duration of conducting of the breathable gases to the inhalation unit 220. The communication unit 102 converts the input from a user to a signal to the distributor of breathable gases 103 to conduct the breathable gases to the inhalation unit 220 for the predefined duration. The distributor of breathable gases 103 receives breathable gases from the source of breathable gases 10M1, receives the signal from the communication unit 102 to conduct the breathable gases to the inhalation unit 220 for the predefined duration, and conducts the breathable gases to the inhalation unit 220 for the predefined duration in response to the signal from the communication unit 102. The inhalation unit 220 receives the breathable gases from the distributor of breathable gases 103 and releases the breathable gases to inhalation organs of a user for the predefined duration. The distributor of breathable gases 103 communicates data of interest, such as remaining duration of conducting of the breathable gases, to the communication unit 102 to be available for a current user. The data of interest can additionally be communicated to a smartphone or a tablet PC of the user.

FIG. 1C illustrates another arrangement of a system configured to supply breathable gases to civilian people. The system comprises a source of liquefied breathable gases 10M3, a liquefied breathable gases dispensing system 100M3 in communication with the source of liquefied breathable gases 10M3 and in temporary communication with a breathing system 200M3. The source of breathable gases 10M3 generates, or stores, or generates and stores, and conducts liquefied oxygen and liquefied inert gas to the liquefied breathable gases dispensing system 100M3. The liquefied breathable gases dispensing system 100M3 receives the liquefied oxygen and liquefied inert gas from the source of liquefied breathable gases 10M3 and dispenses the liquefied oxygen and liquefied inert gas to the breathing system 200M3. In more detail, the liquefied breathable gases dispensing system 100M3 comprises a dispenser of liquefied breathable gases 401 in communication with the source of liquefied breathable gases 10M3 and a communication unit 102 in communication with the dispenser of liquefied breathable gases 401. In more detail, the breathing system 200M1 comprises a container of liquefied oxygen 501A in a temporary communication with the dispenser of liquefied breathable gases 401, a container of liquefied inert gas 501B in a temporary communication with the dispenser of liquefied breathable gases 401, a liquid-to-gas converter 410 in communication with the container of liquefied oxygen 501A and in communication with the container of liquefied inert gas 501B, and an inhalation unit 220 in communication with the liquid-to-gas converter 410. The communication unit 102 receives an input from a user preferably, but not limited to, in a form of a payment transaction, the input defines an amount of the liquefied oxygen and liquefied inert gas to be dispensed by the dispenser of liquefied breathable gases 401 to the container of liquefied oxygen 501A and to the container of liquefied inert gas 501B correspondingly. The communication unit 102 converts the input from a user to a signal to the dispenser of liquefied breathable gases 401 to dispense the predefined amount of liquefied oxygen and liquefied inert gas to the container of liquefied oxygen 501A and to the container of liquefied inert gas 501B correspondingly. The dispenser of liquefied breathable gases 401 receives the liquefied oxygen and liquefied inert gas from the source of liquefied breathable gases 10M3, receives the signal from the communication unit 102 to dispense the predefined amount of liquefied oxygen and liquefied inert gas to the container of liquefied oxygen 501A and to the container of liquefied inert gas 501B correspondingly, and dispenses the predefined amount of liquefied oxygen and liquefied inert gas to the container of liquefied oxygen 501A and to the container of liquefied inert gas 501B correspondingly in response to the signal from the communication unit 102. The dispenser of liquefied breathable gases 401 communicates data of interest, such as the amount of dispensed liquefied oxygen and liquefied inert gas, to the communication unit 102 to be available for a current user. When the predefined amount of liquefied oxygen and liquefied inert gas is dispensed to the container of liquefied oxygen 501A and to the container of liquefied inert gas 501B, the connection between the dispenser of liquefied breathable gases 401 and the container of liquefied oxygen 501A and the connection between the dispenser of liquefied breathable gases 401 and the container of liquefied inert gas 501B should be disconnected, and another container of liquefied oxygen 501A and another container of liquefied inert gas 501B can be connected to the dispenser of breathable gases 401 for dispensing liquefied breathable gases. The liquid-to-gas converter 410 receives the liquefied oxygen and liquefied inert gas from the container of liquefied oxygen 501A and from the container of liquefied inert gas 501B correspondingly, converts the liquefied oxygen and liquefied inert gas to breathable gases, and conducts the breathable gases to the inhalation unit 220. The container of liquefied oxygen 501A and container of liquefied inert gas 501B, containing dispensed liquefied oxygen and liquefied inert gas, disconnected from the dispenser of breathable gases 401, together with the liquid-to-gas converter 410, becomes an autonomous portable source of breathable gases for the inhalation unit 220. The inhalation unit 220 receives the breathable gases from the liquid-to-gas converter 410 and releases the breathable gases to inhalation organs of a user for a duration corresponding to the capacity of the container of liquefied oxygen 501A and the container of liquefied inert gas 501B.

FIG. 1D illustrates another arrangement of a system configured to supply breathable gases to civilian people. The system comprises a source of liquefied breathable gases 10M3, a liquefied breathable gases dispensing system 100M3 in communication with the source of liquefied breathable gases 10M3 and in temporary communication with a breathing system 200M3. The source of breathable gases 10M3 generates, or stores, or generates and stores, and conducts liquefied breathable gases to the liquefied breathable gases dispensing system 100M3. The liquefied breathable gases dispensing system 100M3 receives the liquefied breathable gases from the source of liquefied breathable gases 10M3 and dispenses the liquefied breathable gases to the breathing system 200M3. In more detail, the liquefied breathable gases dispensing system 100M3 comprises a dispenser of liquefied breathable gases 401 in communication with the source of liquefied breathable gases 10M3 and a communication unit 102 in communication with the dispenser of liquefied breathable gases 401. In more detail, the breathing system 200M1 comprises a container of liquefied breathable gases 501 in a temporary communication with the dispenser of liquefied breathable gases 401, a liquid-to-gas converter 410 in communication with the container of liquefied breathable gases 501, and an inhalation unit 220 in communication with the liquid-to-gas converter 410. The communication unit 102 receives an input from a user preferably, but not limited to, in a form of a payment transaction, the input defines an amount of liquefied breathable gases to be dispensed by the dispenser of liquefied breathable gases 401 to the container of liquefied breathable gases 501. The communication unit 102 converts the input from a user to a signal to the dispenser of liquefied breathable gases 401 to dispense the predefined amount of liquefied breathable gases to the container of liquefied breathable gases 501. The dispenser of liquefied breathable gases 401 receives the liquefied breathable gases from the source of liquefied breathable gases 10M3, receives the signal from the communication unit 102 to dispense the predefined amount of liquefied breathable gases to the container of liquefied breathable gases 501, and dispenses the predefined amount of liquefied breathable gases to the container of liquefied breathable gases 501 in response to the signal from the communication unit 102. The dispenser of liquefied breathable gases 401 communicates data of interest, such as amount of dispensed liquefied breathable gases, to the communication unit 102 to be available for a current user. When the predefined amount of liquefied breathable gases is dispensed to the container of liquefied breathable gases 501, the connection between the dispenser of liquefied breathable gases 401 and the container of liquefied breathable gases 501 should be disconnected, and another container of liquefied breathable gases 501 can be connected to the dispenser of breathable gases 401 for dispensing liquefied breathable gases. The liquid-to-gas converter 410 receives the liquefied breathable gases from the container of liquefied breathable gases 501, converts the liquefied breathable gases to breathable gases, and conducts the breathable gases to the inhalation unit 220. The container of liquefied breathable gases 501, containing dispensed liquefied breathable gases, disconnected from the dispenser of breathable gases 401, together with the liquid-to-gas converter 410, becomes an autonomous portable source of breathable gases for the inhalation unit 220. The inhalation unit 220 receives the breathable gases from the liquid-to-gas converter 410 and releases the breathable gases to inhalation organs of a user for a duration corresponding to the capacity of the container of liquefied breathable gases 501.

FIG. 1E illustrates another arrangement of a system configured to supply breathable gases to civilian people. The system comprises a source of liquefied breathable gases 10M3, a liquefied breathable gases dispensing system 100M3 in communication with the source of liquefied breathable gases 10M3 and in temporary communication with a breathing system 200M3. The source of breathable gases 10M3 generates, or stores, or generates and stores, and conducts liquefied oxygen and liquefied inert gas to the liquefied breathable gases dispensing system 100M3. The liquefied breathable gases dispensing system 100M3 receives the liquefied oxygen and liquefied inert gas from the source of liquefied breathable gases 10M3, mixes the received liquefied oxygen and liquefied inert gas in an appropriate proportion so that the mixture becomes liquefied breathable gases, and dispenses the liquefied breathable gases to the breathing system 200M3. In more detail, the liquefied breathable gases dispensing system 100M3 comprises a dispenser of liquefied breathable gases 401 in communication with the source of liquefied breathable gases 10M3 and a communication unit 102 in communication with the dispenser of liquefied breathable gases 401. In more detail, the breathing system 200M1 comprises a container of liquefied breathable gases 501 in a temporary communication with the dispenser of liquefied breathable gases 401, a liquid-to-gas converter 410 in communication with the container of liquefied breathable gases 501, and an inhalation unit 220 in communication with the liquid-to-gas converter 410. The communication unit 102 receives an input from a user preferably, but not limited to, in a form of a payment transaction, the input defines an amount of liquefied breathable gases to be dispensed by the dispenser of liquefied breathable gases 401 to the container of liquefied breathable gases 501. The communication unit 102 converts the input from a user to a signal to the dispenser of liquefied breathable gases 401 to dispense the predefined amount of liquefied breathable gases to the container of liquefied breathable gases 501. The dispenser of liquefied breathable gases 401 receives the liquefied oxygen and liquefied inert gas from the source of liquefied breathable gases 10M3, mixes the liquefied oxygen and liquefied inert gas in an appropriate proportion so that the mixture becomes liquefied breathable gases, receives the signal from the communication unit 102 to dispense the predefined amount of liquefied breathable gases to the container of liquefied breathable gases 501, and dispenses the predefined amount of liquefied breathable gases to the container of liquefied breathable gases 501 in response to the signal from the communication unit 102. The dispenser of liquefied breathable gases 401 communicates data of interest, such as amount of dispensed liquefied breathable gases, to the communication unit 102 to be available for a current user. When the predefined amount of liquefied breathable gases is dispensed to the container of liquefied breathable gases 501, the connection between the dispenser of liquefied breathable gases 401 and the container of liquefied breathable gases 501 should be disconnected, and another container of liquefied breathable gases 501 can be connected to the dispenser of breathable gases 401 for dispensing of the liquefied breathable gases. The liquid-to-gas converter 410 receives the liquefied breathable gases from the container of liquefied breathable gases 501, converts the liquefied breathable gases to breathable gases, and conducts the breathable gases to the inhalation unit 220. The container of liquefied breathable gases 501, containing dispensed liquefied breathable gases, when disconnected from the dispenser of breathable gases 401, together with the liquid-to-gas converter 410, becomes an autonomous portable source of breathable gases for the inhalation unit 220. The inhalation unit 220 receives the breathable gases from the liquid-to-gas converter 410 and releases the breathable gases to inhalation organs of a user for a duration corresponding to the capacity of the container of liquefied breathable gases 501.

FIG. 1F illustrates an arrangement of a source of breathable gases 10M1. The source of breathable gases 10M1 comprises a source of liquefied breathable gases 10M3, and a liquid-to-gas converter 410 in communication with the source of liquefied breathable gases 10M3. The source of liquefied breathable gases 10M3 generates, or stores, or generates and stores, and conducts liquefied oxygen and liquefied inert gas to the liquid-to-gas converter 410. The liquid-to-gas converter 410 receives the liquefied oxygen and liquefied inert gas from the source of liquefied breathable gases 10M3, converts the liquefied oxygen and liquefied inert gas to breathable gases, and conducts the breathable gases to a breathable gases dispensing system 100M1, to a breathable gases distributing system 100M2, or to the both of them.

FIG. 1G illustrates another arrangement of a source of breathable gases 10M1. The source of breathable gases 10M1 comprises a source of liquefied breathable gases 10M3, and a liquid-to-gas converter 410 in communication with the source of liquefied breathable gases 10M3. The source of liquefied breathable gases 10M3 generates, or stores, or generates and stores, and conducts liquefied breathable gases to the liquid-to-gas converter 410. The liquid-to-gas converter 410 receives the liquefied breathable gases from the source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 2 illustrates a dispenser of breathable gases 101 configured to receive breathable gases from a source of breathable gases, dispense the breathable gases to a container of breathable gases in response to a corresponding signal from a communication unit, and control a pressure of the dispensed breathable gases. The dispenser of breathable gases 101 comprises a controllable valve 109 in communication with a source of breathable gases 10M1 by an inlet line and with a container of breathable gases 201 by an outlet line, a pressure sensor 112 in communication with the outlet line of the controllable valve 109, and a controller 110M1 in communication with the controllable valve 109 and with the pressure sensor 112. The controllable valve 109 opens and closes the flow of breathable gases from the source of breathable gases 10M1 to the container of breathable gases 201 in response to corresponding activation signals from the controller 110M1. The pressure sensor 112 continuously measures pressure of the dispensed breathable gases in the line between the controllable valve 109 and the container of breathable gases 201 and sends a corresponding signal to the controller 110M1. The controller 110M1 receives a signal from a communication unit 102 to dispense breathable gases, generates and sends an activation signal to the controllable valve 109 to open the flow of breathable gases from the source of breathable gases 10M1 to the container of breathable gases 201. When pressure of the dispensed breathable gases reaches a predefined level, corresponding to the structural strength of the container of breathable gases 201, the controller 110M1 sends an activation signal to the controllable valve 109 to close the flow of breathable gases. The amount of the dispensed breathable gases can be calculated using a difference between the measured pressure of the dispensed breathable gases at the beginning of dispensing and at the end of dispensing. The controller 110M1 communicates an information of interest, for example, a calculated amount of dispensed breathable gases, to the communication unit 102 to be available for a current user.

FIG. 3 illustrates a dispenser of breathable gases 101 configured to receive breathable gases from a source of breathable gases, dispense the breathable gases to a container of breathable gases in response to a corresponding signal from a communication unit, control the pressure of the dispensed breathable gases, and measure the temperature of the dispensed breathable gases. The dispenser of breathable gases 101 comprises a controllable valve 109 in communication with a source of breathable gases 10M1 by an inlet line and in communication with a container of breathable gases 201 by an outlet line, a pressure sensor 112 in communication with the outlet line of the controllable valve 109, a temperature sensor 111 in communication with the outlet line of the controllable valve 109, and a controller 110M1 in communication with the controllable valve 109, with the temperature sensor 111, and with the pressure sensor 112. The controllable valve 109 opens and closes the flow of breathable gases from the source of breathable gases 10M1 to the container of breathable gases 201 in response to corresponding activation signals from the controller 110M1. The pressure sensor 112 continuously measures the pressure of the dispensed breathable gases in the line between the controllable valve 109 and the container of breathable gases 201, and sends a corresponding signal to the controller 110M1. The temperature sensor 111 measures the temperature of the dispensed breathable gases in the line between the controllable valve 109 and the container of breathable gases 201 and sends a corresponding signal to the controller 110M1 at least at a beginning and at an end of the dispensing of breathable gases. The controller 110M1 receives a signal from a communication unit 102 to dispense breathable gases, generates and sends the activation signal to the controllable valve 109 to open the flow of breathable gases from the source of breathable gases 10M1 to the container of breathable gases 201. When pressure of the dispensed breathable gases reaches a predefined level, corresponding to the structural strength of the container of breathable gases 201, the controller 110M1 sends an activation signal to the controllable valve 109 to close the flow of breathable gases. The amount of the dispensed breathable gases can be calculated using a difference between the measured pressure and the difference between measured temperature of the dispensed breathable gases at the beginning of dispensing and at the end of dispensing. The controller 110M1 communicates an information of interest, for example, a calculated amount of dispensed breathable gases, to the communication unit 102 to be available for a current user.

FIG. 4 illustrates a distributor of breathable gases 103 configured to receive breathable gases from a source of breathable gases and conduct the breathable gases to at least one inhalation unit in response to a corresponding signal from a communication unit. The distributor of breathable gases 103 comprises at least one controllable valve 109M2 in communication with a source of breathable gases 10M1 by an inlet line and in communication with an inhalation unit 220 by an outlet line and a controller 110M2 in communication with the controllable valve 109M2. The controllable valve 109M2 opens and closes the flow of breathable gases from the source of breathable gases 10M1 to the connected inhalation unit 220 in response to corresponding activation signals from the controller 110M2. The controller 110M2 receives a signal from a communication unit 102 to conduct breathable gases to a specified inhalation unit 220 for a predefined duration of time, generates and sends the activation signal to the corresponding controllable valve 109M2 to open the flow of breathable gases from a source of breathable gases 10M1 to the specified inhalation unit 220 for the predefined duration of time, corresponding to the input from a user. When the predefined duration is over, the controller 110M2 sends an activation signal to the controllable valve 109M2 to close the flow of breathable gases. The amount of the distributed breathable gases can be calculated based on the measured duration of the distributing. The controller 110M2 communicates data of interest, such as current remaining duration of conducting of the breathable gases, to the communication unit 102 to be available for a current user. The data of interest can be additionally communicated to a smartphone or a tablet PC of the user.

FIG. 5 illustrates an arrangement of a communication unit 102 configured to receive an input from a user in a form of a payment card payment, communicate the input to a controller, and communicate an information of interest from the controller to the user. The communication unit 102 comprises a card reader 121, a key board 123, and a screen 124. The card reader 121 receives an input from a payment card of a user. The key board 123 receives an input from the user in a form of a sequence of digits and letters to identify, for example, applicability and validity of the user's payment card, and to define the amount of breathable gases to be dispensed, in other words supplied, by the breathable gases dispense system 100M1, the amount of breathable gases to be distributed, in other words supplied, by the breathable gases distributing system 100M2, or the amount of liquefied breathable gases to be dispensed, in other words supplied, by the liquefied breathable gases dispensing system 100M3. The screen 124 indicates information of interest to the user. In general, the communication unit 102 converts the received input from the user to a corresponding signal to the controller 110M1, 110M2, or 110M3 of the breathable gases supply system 100M1, 100M2, or 100M3, correspondingly, to supply a predefined amount of breathable gases. The communication unit 102 receives an information of interest, for example, an amount of breathable gases supplied, from the controller 110M1, 110M2, or 110M3, and indicates the information of interest on the screen 124.

FIG. 6 illustrates another arrangement of a communication unit 102 configured to receive an input from a user in a form of a scan-able bar-code, communicate the input to a controller, and communicate an information of interest from the controller to the user. The communication unit 102 comprises a laser scanner 125, a key board 123, and a screen 124. The key board 123 receives an input from the user in a form of a sequence of digits and letters to identify, for example, applicability and validity of the user's payment card, and to define the amount of breathable gases to be dispensed, in other words supplied, by the breathable gases dispense system 100M1, the amount of breathable gases to be distributed, in other words supplied, by the breathable gases distributing system 100M2, or the amount of liquefied breathable gases to be dispensed, in other words supplied, by the liquefied breathable gases dispensing system 100M3. The laser scanner 125 scans a bar-code 126 and transforms the coded information from the bar-code to a signal to a controller 110M1, 110M2, or 110M3. The bar-code 126 can comprise an information of a payment maid online, and the payment information can be used for the approval of breathable gases supplying. If all the information required to process breathable gases is included in the bar-code, the keyboard 123 is optional. The screen 124 indicates information of interest to the user. In general, the communication unit 102 converts the received input from the user to a corresponding signal to the controller 110M1, 110M2, or 110M3 of the breathable gases supply system 100M1, 100M2, or 100M3, correspondingly, to supply a predefined amount of breathable gases. The communication unit 102 receives an information of interest, for example, an amount of breathable gases supplied, from the controller 110M1, 110M2, or 110M3, and indicates the information of interest on the screen 124.

FIG. 7 illustrates another arrangement of a communication unit 102 configured to receive an input from a user in a form of a scan-able bar-code, receive an input from the user in a form of a payment card payment, communicate the input to a controller, and communicate an information of interest from the controller to the user. The communication unit 102 comprises a card reader 121, a key board 123, a screen 124, and a laser scanner 125. The card reader 121 receives an input from a payment card of a user. The key board 123 receives an input from the user in a form of a sequence of digits and letters to identify, for example, applicability and validity of the user's payment card, and to define the amount of breathable gases to be dispensed, in other words supplied, by the breathable gases dispense system 100M1, the amount of breathable gases to be distributed, in other words supplied, by the breathable gases distributing system 100M2, or the amount of liquefied breathable gases to be dispensed, in other words supplied, by the liquefied breathable gases dispensing system 100M3. The screen 124 indicates information of interest to the user. The laser scanner 125 scans a bar-code 126 and transforms the coded information from the bar-code to a signal to a controller. The bar-code 126 can comprise an information of a payment maid online, and the payment information can be used for the breathable gases supplying approval. In general, the communication unit 102 converts the received input from the user to a corresponding signal to the controller 110M1, 110M2, or 110M3 of the breathable gases supply system 100M1, 100M2, or 100M3, correspondingly, to supply a predefined amount of breathable gases. The communication unit 102 receives an information of interest, for example, an amount of breathable gases supplied, from the controller 110M1, 110M2, or 110M3, and indicates the information of interest on the screen 124.

FIG. 8A illustrates a connected state of a dispenser of breathable gases 101 and a container of breathable gases 201. In this state the dispensing of breathable gases from a source of breathable gases 10M1 to the container of breathable gases 201 occurs when a corresponding signal received from a communication unit 102. A flexible hose 198 is connected by one end to the dispenser of breathable gases 101 and by other end to the container of breathable gases 201 by means of a quick-release connection 300, that makes the process of connecting and disconnecting convenient and fast. Quick-release connection comprises a female connection end 303, that is mounted to an end of the flexible hose 198, and a male connection end 302, that is mounted to the container of breathable gases 201. One direction check valve 301 should be used to prevent a release of breathable gases when the quick-release connection 300 is disconnected.

FIG. 8B illustrates a disconnected state of the dispenser of breathable gases 101 and the container of breathable gases 201. As shown, the female connection end 303 is disconnected from the male connection end 302. When the quick-release connection 300 is disconnected, the container of breathable gases 201 can be relocated to any desired location, and the dispenser of breathable gases 101 can be connected to a next container of breathable gases 201 for dispensing breathable gases to it.

FIG. 9A illustrates a breathing system 200M1 configured to store a reasonable amount of breathable gases in a portable source of breathable gases and conduct the breathable gases from the portable source of breathable gases to inhaling organs of a user in response to an input from the user. The breathing system 200M1 comprises a container of breathable gases 201, a preferably male connection end 302 with a one-way check valve 301 of the quick-release connection 300 at an inlet of the container of breathable gases 201, a controllable valve 209 at an outlet line of the container of breathable gases 201, an inhalation unit 220 in communication with the container of breathable gases 201, a pressure sensor 212 in communication with the container of breathable gases 201, a controller 210M1 in communication with the controllable valve 209 and with the pressure sensor 212, and a communicator 802 in communication with the controller 210M1. The container of breathable gases 201 receives breathable gases from a dispenser of breathable gases 101 when in connected state, as shown on FIG. 8A. When a desired amount of breathable gases is dispensed, and the dispenser of breathable gases 101 stops conducting breathable gases, the quick-release connection 300 should be disconnected, as shown on FIG. 8B. The pressure sensor 212 continuously measures pressure of the breathable gases in the container of breathable gases 201 and communicates a corresponding signal to the controller 210M1. The communicator 802 receives an input from the user and converts the input to a corresponding signal to the controller 210M1 to begin conducting breathable gases, to stop conducting breathable gases, or to change a flow rate of the breathable gases from the container of breathable gases 201 to the inhalation unit 220. A data of interest, such as current level of pressure of breathable gases in the container of breathable gases 201, is being communicated from the controller 210M1 to the communicator 802 to be available for the user. A smartphone or a tablet PC with a corresponding software application can be used as the communicator 802. The controller 210M1 receives a signal from the communicator 802 and sends an activating signal to the controllable valve 209 to open the flow of breathable gases, close the flow of breathable gases, or change flow rate of the flow of breathable gases from the container of breathable gases 210 to an inhalation unit 220. The inhalation unit 220 releases the breathable gases to inhaling organs of the user.

FIG. 9B illustrates a breathing system 200M1 configured to store a reasonable amount of breathable gases in a portable source of breathable gases and conduct the breathable gases from the portable source of breathable gases to inhaling organs of a user. The breathing system 200M1 comprises a container of breathable gases 201, a preferably male connection end 302 with a one-way check valve 301 of the quick-release connection 300 at an inlet of the container of breathable gases 201, a manually controllable valve 219 at an outlet line of the container of breathable gases 201, an inhalation unit 220 in communication with the container of breathable gases 201, a pressure sensor 212 in communication with the container of breathable gases 201, and a communicator 802 in preferably wireless communication with the pressure sensor 212. The container of breathable gases 201 receives breathable gases from a dispenser of breathable gases 101 when in connected state, as shown on FIG. 8A. When a desired amount of breathable gases is dispensed, and the dispenser of breathable gases 101 stops conducting breathable gases, the quick-release connection 300 should be disconnected, as shown on FIG. 8B. The pressure sensor 212 continuously measures pressure of the breathable gases in the container of breathable gases 201 and communicates a corresponding signal to the communicator 802. The communicator 802 receives the signal from the pressure sensor 212 and converts the signal to a numerical data describing current level of pressure of breathable gases in the container of breathable gases 201. A smartphone or a tablet PC with a corresponding software application can be used as the communicator 802. The manually controllable valve 219 opens the flow of breathable gases, closes the flow of breathable gases, or changes flow rate of the flow of breathable gases from the container of breathable gases 210 to an inhalation unit 220. The inhalation unit 220 releases the breathable gases to inhaling organs of the user.

FIG. 10A illustrates a breathing system 200M1 configured for use with a bicycle. The breathing system 200M1 comprises all the elements of the configuration shown on FIG. 9B. The breathing system 200M1 is mounted on the bicycle frame, and the breathing system 200M1 supplies the breathable gases to the bicycle driver and a passenger.

FIG. 10B illustrates a breathing system 200M1 configured for use with a motorized vehicle. The breathing system 200M1 comprises all the elements of the configuration shown on FIG. 9B. The breathing system 200M1 is mounted on the motorized vehicle frame, and the breathing system 200M1 supplies the breathable gases to the motorized vehicle driver and a passenger.

FIG. 11 illustrates a breathing system 200M1 configured for use with an automobile. The breathing system 200M1 comprises all the elements of the configuration shown on FIG. 9A. The breathing system 200M1 is mounted on the automobile's frame, and the breathing system 200M1 supplies the breathable gases to the automobile driver and passengers. The controller 210M1 can be integrated into an automobile ventilation and air conditioning system's controller. The breathable gases can be supplied also to a breathing zone, which means that the breathable gases are released into an automobile cabin at a specified location, for example, near by a driver's seat, or near by a select passenger's seat. The direction of the flow of the releasing breathable gases can be also adjusted so that the breathable gases are supplied to inhaling organs as directly as possible. The measures mentioned above can be integrated with the automobile's ventilation and air conditioning system.

FIG. 12 illustrates a breathing system 200M1 configured for use with a residential compartment. The breathing system 200M1 comprises all the elements of the configuration shown on FIG. 9A. The controller 210M1 can be integrated into a compartment's ventilation and air conditioning system's controller. The breathable gases can be supplied also to a breathing zone, which means the breathable gases are released into a compartment's interior at a specified location, for example, near by a group of people, or inside a select room. The direction of the flow of the releasing breathable gases can be also adjusted so that the breathable gases are supplied to inhaling organs as directly as possible. The measures mentioned above can be integrated with the compartment's ventilation and air conditioning system.

FIG. 13 illustrates another example of a breathing system 200M1 configured for use within a residential compartment. The breathing system 200M1 comprises all the elements of the configuration shown on FIG. 12, wherein the container of breathable gases 201 is mounted on a mobile platform 118, so that the container of breathable gases 201 can easily be moved to by a dispenser of breathable gases 101 for dispensing purposes, and back to the compartment when the container of breathable gases 201 is replenished with breathable gases.

FIG. 14 illustrates an arrangement of a source of breathable gases 10M1. The source of breathable gases 10M1 comprises a container of breathable gases 104. The container of breathable gases 104 stores a reasonable amount of breathable gases and conducts the breathable gases to a breathable gases dispensing system 100M1, to a breathable gases distributing system 100M2, or to the both of them.

FIG. 15 illustrates a storage of breathable gases 104. The storage of breathable gases 104 comprises a container of breathable gases 210M1, a shut-off valve 117 at a discharge line of the container of breathable gases 210M1, and a preferably male connection end 302 of a quick-release connection 300 with a one way check valve 301 at an inlet line of the container of breathable gases 210M1. To receive breathable gases from a source of breathable gases the male connection end 301 should be connected to a corresponding female connection end of the quick-release connection 300 at a source of breathable gases. When connected, the breathable gases can be conducted from the source of breathable gases to the container of breathable gases 210M1 until the pressure of breathable gases in the container of breathable gases 210M1 reaches a predefined level, corresponding to the structural strength of the container of breathable gases 201M1. Then the male connection end 302 should be disconnected from the corresponding female connection end of the quick-release connection 300. The one way check valve 301 closes the inlet line of the container of breathable gases 210M1. The container of breathable gases 210M1, now including a reasonable amount of breathable gases, becomes a local source of breathable gases, that can be conducted to a dispenser of breathable gases 101, to a distributor of breathable gases 103, or to the both of them.

FIG. 16 illustrates another arrangement of a source of breathable gases 10M1. The source of breathable gases 10M1 comprises a mobile container of breathable gases 106. The mobile container of breathable gases 106 stores a reasonable amount of breathable gases and conducts the breathable gases to a breathable gases dispensing system 100M1, to a breathable gases distributing system 100M2, or to the both of them.

FIG. 17 illustrates a mobile container of breathable gases 106. The mobile container of breathable gases 106 comprises all the elements of the configuration shown on FIG. 15, and the elements are mounted to a mobile platform 118. The mobile platform 118 allows to relatively easy change a location of the container of breathable gases 201M1.

FIG. 18 illustrates another arrangement of a source of breathable gases 10M1. The source of breathable gases 10M1 comprises a compressor of breathable gases 105. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them.

FIG. 19A illustrates another arrangement of a source of breathable gases 10M1. The source of breathable gases 10M1 comprises a liquid-to-gas converter 410. The liquid-to-gas converter 410 receives liquefied oxygen and liquefied inert gas from a source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 19B illustrates another arrangement of a source of breathable gases 10M1. The source of breathable gases 10M1 comprises a liquid-to-gas converter 410. The liquid-to-gas converter 410 receives liquefied breathable gases from a source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 20A illustrates a liquid-to-gas converter 410 configured to receive liquefied oxygen and liquefied inert gas from a source of liquefied breathable gases, evaporate the received liquefied oxygen, evaporate the received liquefied inert gas, mix the evaporated oxygen and the evaporated inert gas in an appropriate proportion, and supply the mixed breathable gases to a breathable gases consumer. The liquid-to-gas converter 410 comprises an evaporator of liquefied oxygen 413A, an evaporator of liquefied inert gas 413B, a mixer of breathable gases 411 in communication with the evaporator of liquefied oxygen 413A and with the evaporator of liquefied inert gas 413B, a controllable valve 409A in the line of evaporated oxygen, a controllable valve 409B in the line of evaporated inert gas, an oxygen sensor 412 in communication with the outlet line of the mixer of breathable gases 411, and a controller 450 in communication with the controllable valve 409A, with the controllable valve 409B, and with the oxygen sensor 412. The evaporator of liquefied oxygen 413A receives liquefied oxygen from a source of liquefied breathable gases 10M3, from a container of liquefied oxygen 501A, or from both of them, evaporates the liquefied oxygen, and conducts the evaporated oxygen to the mixer of breathable gases 411. The evaporator of liquefied inert gas 413B receives liquefied inert gas from a source of liquefied breathable gases 10M3, from a container of liquefied inert gas 501B, or from both of them, evaporates the liquefied inert gas, and conducts the evaporated inert gas to the mixer of breathable gases 411. The mixer of breathable gases 411 receives the evaporated oxygen from the evaporator of liquefied oxygen 413A, receives the evaporated inert gas from the evaporator of liquefied inert gas 413B, mixes the received evaporated oxygen and the received evaporated inert gas in an appropriate proportion so that the mixture becomes breathable gases, and conducts the breathable gases to a dispenser of breathable gases 101, to a distributor of breathable gases 103, to an inhalation unit 220, or to any combination of the two or three of them. The controller 450 receives a signal from the oxygen sensor 412 corresponding to the concentration of the oxygen in the breathable gases at the exit from the mixer of breathable gases 411, controls the flow rate of the evaporated oxygen through the controllable valve 409A, and controls the flow rate of the evaporated inert gas through the controllable valve 409B to keep the concentration of oxygen in breathable gases in a predefined range, for example, from 20% vol to 22% vol.

FIG. 20B illustrates a liquid-to-gas converter 410 configured to receive liquefied breathable gases from a source of liquefied breathable gases, evaporate the received liquefied breathable gases, and supply the evaporated breathable gases to a breathable gases consumer. The liquid-to-gas converter 410 comprises an evaporator of liquefied breathable gases 413. The evaporator of liquefied breathable gases 413 receives liquefied breathable gases from a source of liquefied breathable gases 10M3, from a container of liquefied breathable gases 501, or from both of them, evaporates the liquefied breathable gases, and conducts the evaporated breathable gases to a dispenser of breathable gases 101, to a distributor of breathable gases 103, to an inhalation unit 220, or to any combination of the two or three of them.

FIG. 21 illustrates another arrangement of a liquid-to-gas converter 410 configured to receive liquefied oxygen and liquefied inert gas from a source of liquefied breathable gases, evaporate the received liquefied oxygen, evaporate the received liquefied inert gas, mix the evaporated oxygen and the evaporated inert gas in an appropriate proportion, and supply the mixed breathable gases to a breathable gases consumer. The liquid-to-gas converter 410 comprises an evaporator of liquefied oxygen 413A, an evaporator of liquefied inert gas 413B, a mixer of breathable gases 411 in communication with the evaporator of liquefied oxygen 413A and with the evaporator of liquefied inert gas 413B, a controllable valve 409A and a flow rate meter 416A in the line of evaporated oxygen, a controllable valve 409B and a flow rate meter 416B in the line of evaporated inert gas, an oxygen sensor 412 in communication with the outlet line of the mixer of breathable gases 411, and a controller 450 in communication with the controllable valve 409A, with the controllable valve 409B, with the flow rate meter 416A, with the flow rate meter 416B, and with the oxygen sensor 412. The evaporator of liquefied oxygen 413A receives the liquefied oxygen from a source of liquefied breathable gases 10M3, from a container of liquefied oxygen 501A, or from both of them, evaporates the liquefied oxygen, and conducts the evaporated oxygen to the mixer of breathable gases 411. The evaporator of liquefied inert gas 413B receives the liquefied inert gas from a source of liquefied breathable gases 10M3, from a container of liquefied inert gas 501B, or from both of them, evaporates the liquefied inert gas, and conducts the evaporated inert gas to the mixer of breathable gases 411. The mixer of breathable gases 411 receives the evaporated oxygen from the evaporator of liquefied oxygen 413A, receives the evaporated inert gas from the evaporator of liquefied inert gas 413B, mixes the received evaporated oxygen and the received evaporated inert gas so that the mixture becomes breathable gases, and conducts the breathable gases to a dispenser of breathable gases 101, to a distributor of breathable gases 103, to an inhalation unit 220, or to any combination of the two or three of them. The controller 450 receives a signal from the oxygen sensor 412 corresponding to the concentration of the oxygen in the breathable gases at the exit from the mixer of breathable gases 411, receives the signal from the flow rate meter 416A, receives the signal from the flow rate meter 416B, controls the flow rate of the evaporated oxygen through the controllable valve 409A, and controls the flow rate of the evaporated inert gas through the controllable valve 409B to keep the concentration of the oxygen in breathable gases in a predefined range, for example, from 20% vol to 22% vol.

FIG. 22 illustrates another arrangement of a liquid-to-gas converter 410 configured to receive liquefied oxygen and liquefied inert gas from a source of liquefied breathable gases, mix the liquefied oxygen and the liquefied inert gas in an appropriate proportion, evaporate the mixture of the liquefied oxygen and the liquefied inert gas, and supply the evaporated breathable gases to a breathable gases consumer. The liquid-to-gas converter 410 comprises a mixer of liquefied breathable gases 421, a controllable valve 429A in the line of liquefied oxygen, a controllable valve 429B in the line of liquefied inert gas, an evaporator of liquefied breathable gases 413 in communication with the mixer of liquefied breathable gases, an oxygen sensor 412 in communication with the outlet line of the evaporator of liquefied breathable gases 413, and a controller 450 in communication with the controllable valve 429A, with the controllable valve 429B, and with the oxygen sensor 412. The mixer of liquefied breathable gases 421 receives the liquefied oxygen from a source of liquefied breathable gases 10M3, from a container of liquefied oxygen 501A, or from both of them, receives the liquefied inert gas from a source of liquefied breathable gases 10M3, from a container of liquefied inert gas 501B, or from both of them, mixes the received liquefied oxygen and the received liquefied inert gas, and conducts the mixture of liquefied breathable gases to the evaporator of liquefied breathable gases 413. The evaporator of liquefied breathable gases 413 receives the mixture of liquefied breathable gases from the mixer of liquefied breathable gases 421, evaporates the liquefied breathable gases, and conducts the evaporated breathable gases to a dispenser of breathable gases 101, to a distributor of breathable gases 103, to an inhalation unit 220, or to any combination of the two or three of them. The controller 450 receives a signal from the oxygen sensor 412 corresponding to the concentration of the oxygen in the breathable gases at the exit from the evaporator of liquefied breathable gases 413, controls the flow rate of the liquefied oxygen through the controllable valve 429A, and controls the flow rate of the liquefied inert gas through the controllable valve 429B to keep the concentration of oxygen in breathable gases in a predefined range, for example from 20% vol to 22% vol.

FIG. 23 illustrates another arrangement of a liquid-to-gas converter 410 configured to receive liquefied oxygen and liquefied inert gas from a source of liquefied breathable gases, mix the liquefied oxygen and the liquefied inert gas in an appropriate proportion, evaporate the mixture of the liquefied oxygen and the liquefied inert gas, and supply the evaporated breathable gases to a breathable gases consumer. The liquid-to-gas converter 410 comprises a mixer of liquefied breathable gases 421, a controllable valve 429A and a flow rate meter 426A in the line of liquefied oxygen, a controllable valve 429B and a flow rate meter 426B in the line of liquefied inert gas, an evaporator of liquefied breathable gases 413 in communication with the mixer of liquefied breathable gases, an oxygen sensor 412 in communication with the outlet line of the evaporator of liquefied breathable gases 413, and a controller 450 in communication with the controllable valve 429A, with the controllable valve 429B, with the flow rate meter 426A, with the flow rate meter 426B, and with the oxygen sensor 412. The mixer of liquefied breathable gases 421 receives the liquefied oxygen from a source of liquefied breathable gases 10M3, from a container of liquefied oxygen 501A, or from both of them, receives the liquefied inert gas from a source of liquefied breathable gases 10M3, from a container of liquefied inert gas 501B, or from both of them, mixes the received liquefied oxygen and the received liquefied inert gas, and conducts the mixture of liquefied breathable gases to the evaporator of liquefied breathable gases 413. The evaporator of liquefied breathable gases 413 receives the mixture of liquefied breathable gases from the mixer of liquefied breathable gases 421, evaporates the liquefied breathable gases, and conducts the evaporated breathable gases to a dispenser of breathable gases 101, to a distributor of breathable gases 103, to an inhalation unit 220, or to any combination of the two or three of them. The controller 450 receives a signal from the oxygen sensor 412 corresponding to the concentration of the oxygen in the breathable gases at the exit from the evaporator of liquefied breathable gases 413, receives a signal from the flow rate meter 426A, receives a signal from the flow rate meter 426B, controls the flow rate of the liquefied oxygen through the controllable valve 429A, and controls the flow rate of the liquefied inert gas through the controllable valve 429B to keep the concentration of oxygen in breathable gases in a predefined range, for example, from 20% vol to 22% vol.

FIG. 24 illustrates another arrangement of a source of breathable gases 10M1. The source of breathable gases 10M1 comprises a compressor of breathable gases 105 and a mobile container of breathable gases 106. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them. The mobile container of breathable gases 106 stores a reasonable amount of breathable gases and conducts the breathable gases to a breathable gases dispensing system 100M1, to a breathable gases distributing system 100M2, or to the both of them.

FIG. 25A illustrates another arrangement of a source of breathable gases 10M1. The source of breathable gases 10M1 comprises a compressor of breathable gases 105 and a liquid-to-gas converter 410. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to a breathable gases dispensing system 100M1, to a breathable gases distributing system 100M2, or to the both of them.

FIG. 25B illustrates another arrangement of a source of breathable gases 10M1. The source of breathable gases 10M1 comprises a compressor of breathable gases 105 and a liquid-to-gas converter 410. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 26A illustrates another arrangement of a source of breathable gases 10M1. The source of breathable gases 10M1 comprises a mobile container of breathable gases 106 and a liquid-to-gas converter 410. The mobile container of breathable gases 106 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to a breathable gases dispensing system 100M1, to a breathable gases distributing system 100M2, or to the both of them.

FIG. 26B illustrates another arrangement of a source of breathable gases 10M1. The source of breathable gases 10M1 comprises a mobile container of breathable gases 106 and a liquid-to-gas converter 410. The mobile container of breathable gases 106 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 27A illustrates another arrangement of a source of breathable gases 10M1. The source of breathable gases 10M1 comprises a storage of breathable gases 104 and a liquid-to-gas converter 410 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to the storage of breathable gases 104, to a breathable gases dispensing system 100M1, to a breathable gases distributing system 100M2, or to any combination of the two or three of them.

FIG. 27B illustrates another arrangement of a source of breathable gases 10M1. The source of breathable gases 10M1 comprises a storage of breathable gases 104 and a liquid-to-gas converter 410 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them.

FIG. 28A illustrates another arrangement of a source of breathable gases 10M1. The source of breathable gases 10M1 comprises a compressor of breathable gases 105, a mobile container of breathable gases 106, and a liquid-to-gas converter 410. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them. The mobile container of breathable gases 106 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to a breathable gases dispensing system 100M1, to a breathable gases distributing system 100M2, or to the both of them.

FIG. 28B illustrates another arrangement of a source of breathable gases 10M1. The source of breathable gases 10M1 comprises a compressor of breathable gases 105, a mobile container of breathable gases 106, and a liquid-to-gas converter 410. The compressor of breathable gases 105 receives breathable gases from a source of breathable gases of a relatively low pressure, for example, from ambient air, compresses the breathable gases, and conducts the breathable gases to a breathable gases dispensing system 100M1, to a breathable gases distributing system 100M2, or to the both of them. The mobile container of breathable gases 106 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to a breathable gases dispensing system 100M1, to a breathable gases distributing system 100M2, or to the both of them.

FIG. 29 illustrates another arrangement of a source of breathable gases 10M1. The source of breathable gases 10M1 comprises a storage of breathable gases 104 and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 30 illustrates another arrangement of a source of breathable gases 10M1. The source of breathable gases 10M1 comprises a storage of breathable gases 104 and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 31 illustrates another arrangement of a source of breathable gases 10M1. The source of breathable gases 10M1 comprises a storage of breathable gases 104, a compressor of breathable gases 105 in communication with the storage of breathable gases 104, and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 32A illustrates another arrangement of a source of breathable gases 10M1. The source of breathable gases 10M1 comprises a storage of breathable gases 104, a liquid-to-gas converter 410 in communication with the storage of breathable gases 104, and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to the storage of breathable gases 104, to a breathable gases dispensing system 100M1, to a breathable gases distributing system 100M2, or to any combination of the two or three of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 32B illustrates another arrangement of a source of breathable gases 10M1. The source of breathable gases 10M1 comprises a storage of breathable gases 104, a liquid-to-gas converter 410 in communication with the storage of breathable gases 104, and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 33A illustrates another arrangement of a source of breathable gases 10M1. The source of breathable gases 10M1 comprises a storage of breathable gases 104, a liquid-to-gas converter 410 in communication with the storage of breathable gases 104, and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 33B illustrates another arrangement of a source of breathable gases 10M1. The source of breathable gases 10M1 comprises a storage of breathable gases 104, a liquid-to-gas converter 410 in communication with the storage of breathable gases 104, and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 34A illustrates another arrangement of a source of breathable gases 10M1. The source of breathable gases 10M1 comprises a storage of breathable gases 104, a liquid-to-gas converter 410 in communication with the storage of breathable gases 104, a compressor of breathable gases 105 in communication with the storage of breathable gases 104, and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 34B illustrates another arrangement of a source of breathable gases 10M1. The source of breathable gases 10M1 comprises a storage of breathable gases 104, a liquid-to-gas converter 410 in communication with the storage of breathable gases 104, a compressor of breathable gases 105 in communication with the storage of breathable gases 104, and a mobile container of breathable gases 106 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them. The mobile container of breathable gases 106 stores a reasonable amount of breathable gases and conducts the breathable gases to the storage of breathable gases 104, to a breathable gases dispensing system 100M1, to a breathable gases distributing system 100M2, or to any combination of the two or three of them.

FIG. 35 illustrates another arrangement of a source of breathable gases 10M1. The source of breathable gases 10M1 comprises a storage of breathable gases 104, a mobile container of breathable gases 106 in communication with the storage of breathable gases 104, and a trans-compressor of breathable gases 108 in the line between the storage of breathable gases 104 and the mobile container of breathable gases 106. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them.

FIG. 36 illustrates another arrangement of a source of breathable gases 10M1. The source of breathable gases 10M1 comprises a storage of breathable gases 104, a mobile container of breathable gases 106 in communication with the storage of breathable gases 104, a trans-compressor of breathable gases 108 in the line between the storage of breathable gases 104 and the mobile container of breathable gases 106, and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 37A illustrates another arrangement of a source of breathable gases 10M1. The source of breathable gases 10M1 comprises a storage of breathable gases 104, a mobile container of breathable gases 106 in communication with the storage of breathable gases 104, a trans-compressor of breathable gases 108 in the line between the storage of breathable gases 104 and the mobile container of breathable gases 106, and a liquid-to-gas converter 410 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them. The liquid-to-gas converter 410 receives liquefied oxygen and liquefied inert gas from a source of liquefied breathable gases 10M3, converts the liquefied oxygen and liquefied inert gas to breathable gases, and conducts the breathable gases to the storage of breathable gases 104, to a breathable gases dispensing system 100M1, to a breathable gases distributing system 100M2, or to any combination of the two or three of them.

FIG. 37B illustrates another arrangement of a source of breathable gases 10M1. The source of breathable gases 10M1 comprises a storage of breathable gases 104, a mobile container of breathable gases 106 in communication with the storage of breathable gases 104, a trans-compressor of breathable gases 108 in the line between the storage of breathable gases 104 and the mobile container of breathable gases 106, and a liquid-to-gas converter 410 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them.

FIG. 38A illustrates another arrangement of a source of breathable gases 10M1. The source of breathable gases 10M1 comprises a storage of breathable gases 104, a mobile container of breathable gases 106 in communication with the storage of breathable gases 104, a trans-compressor of breathable gases 108 in the line between the storage of breathable gases 104 and the mobile container of breathable gases 106, a liquid-to-gas converter 410 in communication with the storage of breathable gases 104, and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 38B illustrates another arrangement of a source of breathable gases 10M1. The source of breathable gases 10M1 comprises a storage of breathable gases 104, a mobile container of breathable gases 106 in communication with the storage of breathable gases 104, a trans-compressor of breathable gases 108 in the line between the storage of breathable gases 104 and the mobile container of breathable gases 106, a liquid-to-gas converter 410 in communication with the storage of breathable gases 104, and a compressor of breathable gases 105 in communication with the storage of breathable gases 104. The storage of breathable gases 104 stores a reasonable amount of breathable gases and conducts the breathable gases to a breathable gases dispensing system 100M1, to a breathable gases distributing system 100M2, or to the both of them. The trans-compressor of breathable gases 108 receives the breathable gases from the mobile container of breathable gases 106, decreases pressure of the received breathable gases if it is higher than a maximum required pressure in the storage of breathable gases 104, or increases pressure of the received breathable gases if it is lower than a minimum required pressure in the storage of breathable gases 104, and conducts the breathable gases to the storage of breathable gases 104. The mobile container of breathable gases 106 stores a reasonable amount of breathable gases and conducts the breathable gases to the storage of breathable gases 104 by means of the trans-compressor of breathable gases 108, to a breathable gases dispensing system 100M1, to a breathable gases distributing system 100M2, or to any combination of the two or three of them. The liquid-to-gas converter 410 receives liquefied breathable gases from a source of liquefied breathable gases 10M3, converts the liquefied breathable gases to breathable gases, and conducts the breathable gases to the storage of breathable gases 104, to a breathable gases dispensing system 100M1, to a breathable gases distributing system 100M2, or to any combination of the two or three of them. The compressor of breathable gases 105 receives breathable gases from a source of breathable gases of a relatively low pressure, for example, from ambient air, compresses the breathable gases, and conducts the breathable gases to the storage of breathable gases 104, to a breathable gases dispensing system 100M1, to a breathable gases distributing system 100M2, or to any combination of the two or three of them.

FIG. 39A illustrates a dispenser of liquefied breathable gases 401 configured to receive liquefied breathable gases from a source of liquefied breathable gases, dispense the liquefied oxygen to a container of liquefied oxygen and liquefied inert gas to a container of liquefied inert gas in a response to an input from a user, and control the flow rate of liquefied oxygen and liquefied inert gas. The dispenser of breathable gases 401 comprises a controllable valve 509A in communication with a liquefied oxygen line of a source of liquefied breathable gases 10M3, a controllable valve 509B in communication with a liquefied inert gas line of the source of liquefied breathable gases 10M3, a flow rate meter 513A in the outlet line of the controllable valve 509A, a flow rate meter 513B in the outlet line of the controllable valve 509B, and a controller 110M3 in communication with the controllable valve 509A, with the controllable valve 509B, with the flow rate meter 513A, and with the flow rate meter 513B. The controller 110M3 receives a signal from a communication unit 102 to dispense a predefined amount of the liquefied oxygen and a predefined amount of the liquefied inert gas, generates and sends an activation signal to the controllable valve 509A to open the flow of liquefied oxygen from a source of liquefied breathable gases 10M3 to a container of liquefied oxygen 501A, generates and sends an activation signal to the controllable valve 509B to open the flow of liquefied inert gas from a source of liquefied breathable gases 10M3 to a container of liquefied inert gas 501B. When the predefined amount of the liquefied oxygen and the predefined amount of the liquefied inert gas are dispensed to the containers of the liquefied breathable gases 501A and 501B correspondingly, the controller 110M3 sends corresponding activation signals to the controllable valves 509A and 509B to close the flow of the liquefied breathable gases. The controller 110M3 communicates an information of interest, for example, the amount of dispensed liquefied breathable gases, to the communication unit 102.

FIG. 39B illustrates a dispenser of liquefied breathable gases 401 configured to receive liquefied breathable gases from a source of liquefied breathable gases, dispense the liquefied breathable gases to a container of liquefied breathable gases in a response to an input from a user, and control the flow rate of liquefied breathable gases. The dispenser of breathable gases 401 comprises a controllable valve 509 in communication with a liquefied breathable gases line of a source of liquefied breathable gases 10M3, a flow rate meter 513 in the outlet line of the controllable valve 509, and a controller 110M3 in communication with the controllable valve 509 and with the flow rate meter 513. The controller 110M3 receives a signal from a communication unit 102 to dispense a predefined amount of the liquefied breathable gases, generates and sends an activation signal to the controllable valve 509 to open the flow of liquefied breathable gases from a source of liquefied breathable gases 10M3 to a container of liquefied breathable gases 501. When the predefined amount of the liquefied breathable gases is dispensed to the container of liquefied breathable gases 501, the controller 110M3 sends a corresponding activation signal to the controllable valve 509 to close the flow of the liquefied breathable gases. The controller 110M3 communicates an information of interest, for example, the amount of dispensed liquefied breathable gases, to the communication unit 102.

FIG. 39C illustrates a dispenser of liquefied breathable gases 401 configured to receive liquefied oxygen and liquefied inert gas from a source of liquefied breathable gases, mix the liquefied oxygen and the liquefied inert gas in an appropriate proportion, and dispense the liquefied breathable gases to a container of liquefied breathable gases in a response to an input from a user. The dispenser of breathable gases 401 comprises a controllable valve 509A in communication with a liquefied oxygen line of a source of liquefied breathable gases 10M3, a controllable valve 509B in communication with a liquefied inert gas line of the source of liquefied breathable gases 10M3, a flow rate meter 513A in the outlet line of the controllable valve 509A, a flow rate meter 513B in the outlet line of the controllable valve 509B, a mixer of liquefied breathable gases 421 in communication with the liquefied oxygen line and in communication with the liquefied inert gas line, and a controller 110M3 in communication with the controllable valve 509A, with the controllable valve 509B, with the flow rate meter 513A, and with the flow rate meter 513B. The controller 110M3 receives a signal from a communication unit 102 to dispense a predefined amount of the liquefied breathable gases, generates and sends an activation signal to the controllable valve 509A to open the flow of liquefied oxygen from a source of liquefied breathable gases 10M3 to the mixer of liquefied breathable gases 421, generates and sends an activation signal to the controllable valve 509B to open the flow of liquefied inert gas from a source of liquefied breathable gases 10M3 to the mixer of liquefied breathable gases 421. The mixer of liquefied breathable gases 421 receives the liquefied oxygen and the liquefied inert gas, mixes them to liquefied breathable gases, and conducts the liquefied breathable gases to a container of liquefied breathable gases 501. When the predefined amount of the liquefied breathable gases is dispensed to the container of liquefied breathable gases 501, the controller 110M3 sends corresponding activation signals to the controllable valves 509A and 509B to close the flow of the liquefied oxygen and liquefied inert gas correspondingly. The controller 110M3 communicates an information of interest, for example, the amount of dispensed liquefied breathable gases, to the communication unit 102.

FIG. 39D illustrates a dispenser of liquefied breathable gases 401 configured to receive liquefied oxygen and liquefied inert gas from a source of liquefied breathable gases, mix the liquefied oxygen and the liquefied inert gas in an appropriate proportion, store a reasonable amount of the liquefied breathable gases, and dispense the liquefied breathable gases to a container of liquefied breathable gases in a response to an input from a user. The dispenser of breathable gases 401 comprises a controllable valve 509A in communication with a liquefied oxygen line of a source of liquefied breathable gases 10M3, a controllable valve 509B in communication with a liquefied inert gas line of the source of liquefied breathable gases 10M3, a flow rate meter 513A in the outlet line of the controllable valve 509A, a flow rate meter 513B in the outlet line of the controllable valve 509B, a mixer of liquefied breathable gases 421 in communication with the liquefied oxygen line and in communication with the liquefied inert gas line, a container of liquefied breathable gases 501M3 in communication with the outlet line of the mixer of breathable gases 421, a controllable valve 509 in communication with the container of liquefied breathable gases 501M3, a flow rate meter 513 in the outlet line of the controllable valve 509, and a controller 110M3 in communication with the controllable valve 509A, with the controllable valve 509B, with the flow rate meter 513A, with the flow rate meter 513B, with the controllable valve 509, and with the flow meter 513. The controller 110M3 receives a signal from a communication unit 102 to dispense a predefined amount of liquefied breathable gases, generates and sends an activation signal to the controllable valve 509 to open the flow of liquefied breathable gases from the container of liquefied breathable gases 501M3 to a container of liquefied breathable gases 501. When the predefined amount of the liquefied breathable gases is dispensed to the container of liquefied breathable gases 501, the controller 110M3 sends a corresponding activation signal to the controllable valve 509 to close the flow of the liquefied breathable gases. To replenish the container of liquefied breathable gases 501M3 with liquefied breathable gases, the controller 110M3 sends corresponding activation signals to the controllable valve 509A to open the flow of liquefied oxygen from a source of liquefied breathable gases 10M3 to the mixer of liquefied breathable gases 421, and to the controllable valve 509B to open the flow of liquefied inert gas from a source of liquefied breathable gases 10M3 to the mixer of liquefied breathable gases 421. The mixer of liquefied breathable gases 421 receives the liquefied oxygen from the outlet line of the controllable valve 509A, receives the liquefied inert gas from the outlet line of the controllable valve 509B, mixes the received liquefied oxygen and liquefied inert gas so that the mixture becomes liquefied breathable gases, and conducts the liquefied breathable gases to a container of liquefied breathable gases 501M3. The controller 110M3 communicates an information of interest, for example, the amount of dispensed liquefied breathable gases, to the communication unit 102.

FIG. 40A illustrates a connected state of the dispenser of liquefied breathable gases 401, a container of liquefied oxygen 501A, and a container of liquefied inert gas 501B. A flexible hose 598A is connected by one end to the dispenser of breathable gases 401 and by other end to the container of liquefied oxygen 501A by means of a quick-release connection 700A. The quick-release connection 700A comprises a female connection end 703A, that is mounted to an end of the flexible hose 598A, and a male connection end 702A, that is mounted to the container of liquefied oxygen 501A. One direction check valve 701A should be used to prevent a release of the liquefied oxygen when the quick-release connection 700A is disconnected. A flexible hose 598B is connected by one end to the dispenser of breathable gases 401 and by other end to the container of liquefied inert gas 501B by means of a quick-release connection 700B. The quick-release connection 700B comprises a female connection end 703B, that is mounted to an end of the flexible hose 598B, a male connection end 702B, that is mounted to the container of liquefied inert gas 501B. One direction check valve 701B should be used to prevent a release of the liquefied inert gas when the quick-release connection 700B is disconnected.

FIG. 40B illustrates a disconnected state of the dispenser of liquefied breathable gases 401, a container of liquefied oxygen 501A, and a container of liquefied inert gas 501B. As shown, the female connection end 703A is disconnected from the male connection end 702A, and the female connection end 703B is disconnected from the male connection end 702B. When the quick-release connections 700A and 700B are disconnected, the container of liquefied oxygen 501A and the container of liquefied inert gas 501B can be relocated to any desired location, and the dispenser of liquefied breathable gases 401 can be connected to a next container of liquefied oxygen 501A and to a next container of liquefied inert gas 501B for dispensing liquefied breathable gases to them.

FIG. 40C illustrates a connected state of the dispenser of liquefied breathable gases 401 and a container of liquefied breathable gases 501. A flexible hose 598 is connected by one end to the dispenser of breathable gases 401 and by other end to the container of liquefied breathable gases 501 by means of a quick-release connection 700. The quick-release connection 700 comprises a female connection end 703, that is mounted to an end of the flexible hose 598, and a male connection end 702, that is mounted to the container of liquefied breathable gases 501. One direction check valve 701 should be used to prevent a release of the liquefied breathable gases when the quick-release connection 700 is disconnected.

FIG. 40D illustrates a disconnected state of the dispenser of liquefied breathable gases 401 and a container of liquefied breathable gases 501. As shown, the female connection end 703 is disconnected from the male connection end 702. When the quick-release connection 700 is disconnected, the container of liquefied breathable gases 501 can be relocated to any desired location, and the dispenser of liquefied breathable gases 401 can be connected to a next container of liquefied breathable gases 501 for dispensing liquefied breathable gases to it.

FIG. 40E illustrates a connected state of the dispenser of liquefied breathable gases 401 and a container of liquefied breathable gases 501. A flexible hose 598 is connected by one end to the dispenser of breathable gases 401 and by other end to the container of liquefied breathable gases 501 by means of a quick-release connection 700. The quick-release connection 700 comprises a female connection end 703, that is mounted to an end of the flexible hose 598, and a male connection end 702, that is mounted to the container of liquefied breathable gases 501. One direction check valve 701 should be used to prevent a release of the liquefied breathable gases when the quick-release connection 700 is disconnected.

FIG. 40F illustrates a disconnected state of the dispenser of liquefied breathable gases 401 and a container of liquefied breathable gases 501. As shown, the female connection end 703 is disconnected from the male connection end 702. When the quick-release connection 700 is disconnected, the container of liquefied breathable gases 501 can be relocated to any desired location, and the dispenser of liquefied breathable gases 401 can be connected to a next container of liquefied breathable gases 501 for dispensing liquefied breathable gases to it.

FIG. 41A illustrates an arrangement of a source of liquefied breathable gases 10M3. The source of liquefied breathable gases 10M3 comprises a storage of liquefied oxygen 504A, and a storage of liquefied inert gas 504B. The storage of liquefied oxygen 504A stores a reasonable amount of liquefied oxygen and conducts the liquefied oxygen to a liquid-to-gas converter 410, to a liquefied breathable gases dispensing system 100M3, or to the both of them. The storage of liquefied inert gas 504B stores a reasonable amount of liquefied inert gas and conducts the liquefied inert gas to a liquid-to-gas converter 410, to a liquefied breathable gases dispensing system 100M3, or to the both of them.

FIG. 41B illustrates an arrangement of a source of liquefied breathable gases 10M3. The source of liquefied breathable gases 10M3 comprises a storage of liquefied oxygen 504. The storage of liquefied breathable gases 504 stores a reasonable amount of liquefied breathable gases and conducts the liquefied breathable gases to a liquid-to-gas converter 410, to a liquefied breathable gases dispensing system 100M3, or to the both of them.

FIG. 42A illustrates a storage of liquefied oxygen 504A and a storage of liquefied inert gas 504B. The storage of liquefied oxygen 504A comprises a container of liquefied oxygen 501M3A, a shut-off valve 517A at an outlet line of the container of liquefied oxygen 501M3A, and a preferably male connection end 702A of a quick-release connection 700A with a one way check valve 701A at an inlet line of the container of liquefied oxygen 501M3A. To receive liquefied oxygen from a source of liquefied oxygen the male connection end 701A should be connected to a corresponding female connection end of the quick-release connection 700A at a source of liquefied oxygen. When connected, the liquefied oxygen can be conducted from a source of liquefied oxygen to the container of liquefied oxygen 501M3A until the level of liquefied oxygen in the container of liquefied oxygen 501M3A reaches a predefined range corresponding to the capacity of the container of liquefied oxygen 501M3A. Then the male connection end 702A should be disconnected from the corresponding female connection end of the quick-release connection 700A. The one way check valve 701A prevents backflow from the inlet line of the container of liquefied oxygen 501M3A. The storage of liquefied inert gas 504B comprises a container of liquefied inert gas 501M3B, a shut-off valve 517B at an outlet line of the container of liquefied inert gas 501M3B, and a preferably male connection end 702B of a quick-release connection 700B with a one way valve 701B at an inlet line of the liquefied inert gas 501M3B. To receive liquefied inert gas from a source of liquefied inert gas the male connection end 701B should be connected to a corresponding female connection end of the quick-release connection 700B at a source of liquefied inert gas. When connected, the liquefied inert gas can be conducted from a source of liquefied inert gas to the container of liquefied inert gas 501M3B until the level of liquefied inert gas in the container of liquefied inert gas 501M3B reaches a predefined range corresponding to the capacity of the container of liquefied inert gas 501M3B. Then the male connection end 702B should be disconnected from the corresponding female connection end of the quick-release connection 700B. The one way check valve 701B prevents backflow from the inlet line of the container of liquefied inert gas 501M3B. Both containers now, including a reasonable amount of the liquefied oxygen and a reasonable amount of the liquefied inert gas, become a local source of liquefied breathable gases for a liquid-to-gas converter 410, for a dispenser of liquefied breathable gases 401, or for both of them.

FIG. 42B illustrates a storage of liquefied breathable gases 504. The storage of liquefied breathable gases 504 comprises a container of liquefied breathable gases 501M3, a shut-off valve 517 at an outlet line of the container of liquefied breathable gases 501M3, and a preferably male connection end 702 of a quick-release connection 700 with a one way check valve 701 at an inlet line of the container of liquefied breathable gases 501M3. To receive liquefied breathable gases from a source of liquefied oxygen the male connection end 701 should be connected to a corresponding female connection end of the quick-release connection 700 at a source of liquefied breathable gases. When connected, the liquefied breathable gases can be conducted from a source of liquefied breathable gases to the container of liquefied breathable gases 501M3 until the level of liquefied breathable gases in the container of liquefied breathable gases 501M3 reaches a predefined range corresponding to the capacity of the container of liquefied breathable gases 501M3. Then the male connection end 702 should be disconnected from the corresponding female connection end of the quick-release connection 700. The one way check valve 701 closes the inlet line of the container of liquefied breathable gases 501M3. The container of liquefied breathable gases now, including a reasonable amount of the liquefied breathable gases, becomes a local source of liquefied breathable gases for a liquid-to-gas converter 410, for a dispenser of liquefied breathable gases 401, or for both of them.

FIG. 43A illustrates another arrangement of a source of liquefied breathable gases 10M3. The source of liquefied breathable gases 10M3 comprises a mobile container of liquefied oxygen 506A and a mobile container of liquefied inert gas 506B. The mobile container of liquefied oxygen 506A stores a reasonable amount of liquefied oxygen and conducts the liquefied oxygen to a dispenser of liquefied breathable gases 401, to a liquid-to-gas converter 410, or to the both of them. The mobile container of liquefied inert gas 506B stores a reasonable amount of liquefied inert gas and conducts the liquefied inert gas to a liquefied breathable gases dispensing system 100M3, to a liquid-to-gas converter 410, or to the both of them.

FIG. 43B illustrates another arrangement of a source of liquefied breathable gases 10M3. The source of liquefied breathable gases 10M3 comprises a mobile container of liquefied breathable gases 506. The mobile container of liquefied breathable gases 506 stores a reasonable amount of liquefied breathable gases and conducts the liquefied breathable gases to a liquefied breathable gases dispensing system 100M3, to a liquid-to-gas converter 410, or to the both of them.

FIG. 44A illustrates a mobile container of liquefied oxygen 506A, and a mobile container of liquefied inert gas 506B. The mobile container of liquefied oxygen 506A and the mobile container of liquefied inert gas 506B comprise all the elements of the configuration shown on FIG. 42A, and the elements are mounted to a mobile platform 118. The mobile platform 118 allows to relatively easy change a location of the mobile container of liquefied oxygen 506A and the mobile container of liquefied inert gas 506B.

FIG. 44B illustrates a mobile container of liquefied breathable gases 506. The mobile container of liquefied breathable gases 506 comprises all the elements of the configuration shown on FIG. 42B, and the elements are mounted to a mobile platform 118. The mobile platform 118 allows to relatively easy change a location of the mobile container of liquefied breathable gases 506A.

FIG. 45A illustrates another arrangement of a source of liquefied breathable gases 10M3. The source of liquefied breathable gases 10M3 comprises an air separation and liquefaction system 505. The air separation and liquefaction system 505 receives an ambient air, separates oxygen and inert gases, such as nitrogen, helium, and argon, from the received air, liquefies the separated oxygen, liquefies at least one of the separated inert gases, and conducts the liquefied oxygen and the liquefied inert gas to a liquefied breathable gases dispensing system 100M3, to a liquid-to-gas converter 410, or to the both of them.

FIG. 45B illustrates another arrangement of a source of liquefied breathable gases 10M3. The source of liquefied breathable gases 10M3 comprises an air liquefaction system 555. The air liquefaction system 555 receives an ambient air, liquefies the received ambient air to liquefied breathable gases, and conducts the liquefied breathable gases to a liquefied breathable gases dispensing system 100M3, to a liquid-to-gas converter 410, or to the both of them.

FIG. 46A illustrates another arrangement of a source of liquefied breathable gases 10M3. The source of liquefied breathable gases 10M3 comprises a storage of liquefied oxygen 504A, a mobile container of liquefied oxygen 506A in communication with the storage of liquefied oxygen 504A, a storage of liquefied inert gas 504B, and a mobile container of liquefied inert gas 506B in communication with the storage of liquefied inert gas 504B. The storage of liquefied oxygen 504A stores a reasonable amount of liquefied oxygen and conducts the liquefied oxygen to a liquefied breathable gases dispensing system 100M3, to a liquid-to-gas converter 410, or to the both of them. The storage of liquefied inert gas 504B stores a reasonable amount of liquefied inert gas and conducts the liquefied inert gas to a liquefied breathable gases dispensing system 100M3, to a liquid-to-gas converter 410, or to the both of them. The mobile container of liquefied oxygen 506A stores a reasonable amount of liquefied oxygen and conducts the liquefied oxygen to the storage of liquefied oxygen 504A, to a liquid-to-gas converter 410, to a liquefied breathable gases dispensing system 100M3, or to any combination of the two or three of them. The mobile container of liquefied inert gas 506B stores a reasonable amount of liquefied inert gas and conducts the liquefied inert gas to the storage of liquefied inert gas 504B, to a liquid-to-gas converter 410, to a liquefied breathable gases dispensing system 100M3, or to any combination of the two or three of them.

FIG. 46B illustrates another arrangement of a source of liquefied breathable gases 10M3. The source of liquefied breathable gases 10M3 comprises a storage of liquefied breathable gases 504 and a mobile container of liquefied breathable gases 506 in communication with the storage of liquefied breathable gases 504. The storage of liquefied breathable gases 504 stores a reasonable amount of liquefied breathable gases and conducts the liquefied breathable gases to a liquefied breathable gases dispensing system 100M3, to a liquid-to-gas converter 410, or to the both of them. The mobile container of liquefied breathable gases 506 stores a reasonable amount of liquefied breathable gases and conducts the liquefied breathable gases to the storage of liquefied breathable gases 504, to a liquid-to-gas converter 410, to a liquefied breathable gases dispensing system 100M3, or to any combination of the two or three of them.

FIG. 47A illustrates another arrangement of a source of liquefied breathable gases 10M3. The source of liquefied breathable gases 10M3 comprises a storage of liquefied oxygen 504A, a storage of liquefied inert gas 504B, and an air separation and liquefaction system 505 in communication with the storage of liquefied oxygen 504A and with the storage of liquefied inert gas 504B. The storage of liquefied oxygen 504A stores a reasonable amount of liquefied oxygen and conducts the liquefied oxygen to a liquefied breathable gases dispensing system 100M3, to a liquid-to-gas converter 410, or to the both of them. The storage of liquefied inert gas 504B stores a reasonable amount of liquefied inert gas and conducts the liquefied inert gas a liquefied breathable gases dispensing system 100M3, to a liquid-to-gas converter 410, or to the both of them. The air separation and liquefaction system 505 receives an ambient air, separates oxygen and inert gases, such as nitrogen, helium, and argon, from the received air, liquefies the separated oxygen, liquefies at least one of the separated inert gases, and conducts the liquefied oxygen and the liquefied inert gas to the storage of liquefied oxygen 504A and to the storage of liquefied inert gas 504B correspondingly, to a liquid-to-gas converter 410, to a liquefied breathable gases dispensing system 100M3, or to any combination of the two or three of them.

FIG. 47B illustrates another arrangement of a source of liquefied breathable gases 10M3. The source of liquefied breathable gases 10M3 comprises a storage of liquefied breathable gases 504 and an air liquefaction system 555 in communication with the storage of liquefied breathable gases 504. The storage of liquefied breathable gases 504 stores a reasonable amount of liquefied breathable gases and conducts the liquefied breathable gases a liquefied breathable gases dispensing system 100M3, to a liquid-to-gas converter 410, or to the both of them. The air liquefaction system 555 receives an ambient air, liquefies the received ambient air to liquefied breathable gases, and conducts the liquefied breathable gases to the storage of liquefied breathable gases 504, to a liquefied breathable gases dispensing system 100M3, to a liquid-to-gas converter 410, or to any combination of the two or three of them.

FIG. 48A illustrates another arrangement of a source of liquefied breathable gases 10M3. The source of liquefied breathable gases 10M3 comprises a storage of liquefied oxygen 504A, a mobile container of liquefied oxygen 506A in communication with the storage of liquefied oxygen 504A, a storage of liquefied inert gas 504B, a mobile container of liquefied inert gas 506B in communication with the storage of liquefied inert gas 504B, and an air separation and liquefaction system 505 in communication with the storage of liquefied oxygen 504A and with the storage of liquefied inert gas 504B. The storage of liquefied oxygen 504A stores a reasonable amount of liquefied oxygen and conducts the liquefied oxygen to a liquefied breathable gases dispensing system 100M3, to a liquid-to-gas converter 410, or to the both of them. The storage of liquefied inert gas 504B stores a reasonable amount of liquefied inert gas and conducts the liquefied inert gas to a liquefied breathable gases dispensing system 100M3, to a liquid-to-gas converter 410, or to the both of them. The mobile container of liquefied oxygen 506A stores a reasonable amount of liquefied oxygen and conducts the liquefied oxygen to the storage of liquefied oxygen 504A, to a liquefied breathable gases dispensing system 100M3, to a liquid-to-gas converter 410, or to any combination of the two or three of them. The mobile container of liquefied inert gas 506B stores a reasonable amount of liquefied inert gas and conducts the liquefied inert gas to the storage of liquefied inert gas 504B, to a liquefied breathable gases dispensing system 100M3, to a liquid-to-gas converter 410, or to any combination of the two or three of them. The air separation and liquefaction system 505 receives an ambient air, separates oxygen and inert gases, such as nitrogen, helium, and argon, from the received air, liquefies the separated oxygen, liquefies at least one of the separated inert gases, and conducts the liquefied oxygen and the liquefied inert gas to the storage of liquefied oxygen 504A and to the storage of liquefied inert gas 504B correspondingly, to a liquefied breathable gases dispensing system 100M3, to a liquid-to-gas converter 410, or to any combination of the two or three of them.

FIG. 48B illustrates another arrangement of a source of liquefied breathable gases 10M3. The source of liquefied breathable gases 10M3 comprises a storage of liquefied breathable gases 504, a mobile container of liquefied breathable gases 506 in communication with the storage of liquefied breathable gases 504, and an air liquefaction system 555 in communication with the storage of liquefied breathable gases 504. The storage of liquefied breathable gases 504 stores a reasonable amount of liquefied breathable gases and conducts the liquefied breathable gases to a liquefied breathable gases dispensing system 100M3, to a liquid-to-gas converter 410, or to the both of them. The mobile container of liquefied breathable gases 506 stores a reasonable amount of liquefied breathable gases and conducts the liquefied breathable gases to the storage of liquefied oxygen 504, to a liquefied breathable gases dispensing system 100M3, to a liquid-to-gas converter 410, or to any combination of the two or three of them. The air liquefaction system 555 receives an ambient air, liquefies the received ambient air to liquefied breathable gases, and conducts the liquefied breathable gases to the storage of liquefied breathable gases 504, to a liquefied breathable gases dispensing system 100M3, to a liquid-to-gas converter 410, or to any combination of the two or three of them.

FIG. 49A illustrates a breathing system 200M3 configured to store a reasonable amount of liquefied breathable gases in a portable source of liquefied breathable gases, evaporate the liquefied breathable gases, and conduct the evaporated breathable gases to an inhalation unit in response to an input from a user. The breathing system 200M3 comprises a container of liquefied oxygen 501A, a preferably male connection end 702A with a one-way check valve 701A of a quick-release connection 700A at an inlet of the container of liquefied oxygen 501A, a container of liquefied inert gas 501B, a preferably male connection end 702B with a one-way check valve 701B of a quick-release connection 700B at an inlet of the container of liquefied inert gas 501B, a liquid-to-gas converter 410 in communication with the container of liquefied oxygen 501A and with the container of liquefied inert gas 501B, a controllable valve 209 at an outlet of the liquid-to-gas converter 410, an inhalation unit 220 in communication with the outlet line of the liquid-to-gas converter 410, a controller 210M3 in communication with the controllable valve 209 and with the liquid-to-gas converter 410, and a communicator 802 in communication with the controller 210M3. The container of liquefied oxygen 501A receives liquefied oxygen, and the container of liquefied inert gas 501B receives liquefied inert gas, from a dispenser of liquefied breathable gases 401 when in connected state, as shown on FIG. 40A. When a desired amount of liquefied breathable gases is dispensed and the dispenser of breathable gases 401 stops conducting liquefied breathable gases the quick-release connections 700A and 700B should be disconnected, as shown on FIG. 40B. After that the container of liquefied oxygen 501A and the container of liquefied inert gas 501B become an autonomous portable source of liquefied breathable gases. The container of liquefied oxygen 501A and the container of liquefied inert gas 501B now contain liquefied breathable gases in the amount corresponding to their capacity, and they can conduct the liquefied oxygen and liquefied inert gas to the liquid-to-gas converter 410. The liquid-to-gas converter 410 receives the liquefied oxygen from the container of liquefied oxygen 501A and the liquefied inert gas from the container of liquefied inert gas 501B, converts the liquefied oxygen and liquefied inert gas to breathable gases, and conducts the breathable gases to the inhalation unit 220. The controller 210M3 receives a signal from a communicator 802 to begin supplying breathable gases to a user, generates and sends a corresponding activating signal to the liquid-to-gas converter 410 to begin converting liquefied breathable gases to breathable gases, generates and sends a corresponding activating signal to the controllable valve 209 to open the flow of breathable gases from the liquid-to-gas converter 410 to the inhalation unit 220. The inhalation unit 220 receives the breathable gases from the liquid-to-gas converter 410 and releases the breathable gases to inhalation organs of a user for a duration corresponding to the capacity of the container of liquefied oxygen 501A and the container of liquefied inert gas 501B. The controller 210M3 also exchanges signals with the liquid-to-gas converter 410, and in whole the controller 210M3 monitors and maintains the concentration of oxygen in the breathable gases in a predefined range, for example from 20% vol to 22% vol. A mobile phone, a tablet PC, or a smart phone with a corresponding software application installed can be used as the communicator 802.

FIG. 49B illustrates a breathing system 200M3 configured to store a reasonable amount of liquefied breathable gases in a portable source of liquefied breathable gases, evaporate the liquefied breathable gases, and conduct the evaporated breathable gases to an inhalation unit in response to an input from a user. The breathing system 200M3 comprises a container of liquefied breathable gases 501, a preferably male connection end 702 with a one-way check valve 701 of a quick-release connection 700A at an inlet of the container of liquefied breathable gases 501, a liquid-to-gas converter 410 in communication with the container of liquefied breathable gases 501, a controllable valve 209 at an outlet of the liquid-to-gas converter 410, an inhalation unit 220 in communication with the liquid-to-gas converter 410, a controller 210M3 in communication with the controllable valve 209 and with the liquid-to-gas converter 410, and a communicator 802 in communication with the controller 210M3. The container of liquefied breathable gases 501 receives liquefied breathable gases from a dispenser of liquefied breathable gases 401 when in connected state, as shown on FIG. 40C and FIG. 40E. When a desired amount of liquefied breathable gases is dispensed and the dispenser of breathable gases 401 stops conducting liquefied breathable gases the quick-release connection 700 should be disconnected, as shown on FIG. 40D and FIG. 40F. After that the container of liquefied breathable gases 501 becomes an autonomous portable source of liquefied breathable gases. The container of liquefied breathable gases 501 now contain liquefied breathable gases in the amount corresponding to its capacity, and it can conduct the liquefied breathable gases to the liquid-to-gas converter 410. The liquid-to-gas converter 410 receives the liquefied breathable gases from the container of liquefied breathable gases 501, converts the liquefied breathable gases to breathable gases, and conducts the breathable gases to the inhalation unit 220. The controller 210M3 receives a signal from a communicator 802 to begin supplying breathable gases to a user, generates and sends a corresponding activating signal to the liquid-to-gas converter 410 to begin converting liquefied breathable gases to breathable gases, generates and sends a corresponding activating signal to the controllable valve 209 to open the flow of breathable gases from the liquid-to-gas converter 410 to the inhalation unit 220. The inhalation unit 220 receives the breathable gases from the liquid-to-gas converter 410 and releases the breathable gases to inhalation organs of a user for a duration corresponding to the capacity of the container of liquefied breathable gases 501. The controller 210M3 also exchanges signals with the liquid-to-gas converter 410, and in whole the controller 210M3 monitors and maintains the concentration of oxygen in the breathable gases in a predefined range, for example from 20% vol to 22% vol. A mobile phone, a tablet PC, or a smart phone with a corresponding software application installed can be used as the communicator 802.

FIG. 50A illustrates breathing system 200M3 configured for use with a bicycle. The breathing system 200M3 comprises all the elements of the configuration shown on FIG. 49A. The elements of the breathing system 200M3 are mounted on the bicycle frame, and the breathing system 200M3 supplies the breathable gases to the bicycle driver and passengers.

FIG. 50B illustrates a breathing system 200M3 configured for use with a motorized vehicle. The breathing system 200M3 comprises the same elements as the configuration shown on FIG. 49A. The elements of the breathing system 200M3 are mounted on the motorized vehicle frame, and the breathing system 200M3 supplies the breathable gases to the motorized vehicle driver and passengers.

FIG. 50C illustrates breathing system 200M3 configured for use with a bicycle. The breathing system 200M3 comprises all the elements of the configuration shown on FIG. 49B. The elements of the breathing system 200M3 are mounted on the bicycle frame, and the breathing system 200M3 supplies the breathable gases to the bicycle driver and passengers.

FIG. 50D illustrates a breathing system 200M3 configured for use with a motorized vehicle. The breathing system 200M3 comprises the same elements as the configuration shown on FIG. 49B. The elements of the breathing system 200M3 are mounted on the motorized vehicle frame, and the breathing system 200M3 supplies the breathable gases to the motorized vehicle driver and passengers.

FIG. 51A illustrates a breathing system 200M3 configured for use with an automobile. The breathing system 200M3 comprises all the elements of the configuration shown on FIG. 49A. The elements of the breathing system 200M3 are mounted on the automobile frame, and the breathing system 200M3 supplies the breathable gases to the automobile driver and passengers. The controller 210M3 can be integrated into the automobile's ventilation and air conditioning system controller. The breathable gases can be supplied to a breathing zone, which means the breathable gases are released into an automobile's cabin at a specified location, for example, near by a driver's seat, or near by a select passenger's seat, or near by the both of them. The direction of the flow of the released breathable gases can be also adjusted so that the breathable gases are supplied to by inhaling organs as directly as possible. The measures mentioned above can be integrated with the automobile's ventilation and air conditioning system.

FIG. 51B illustrates a breathing system 200M3 configured for use with an automobile. The breathing system 200M3 comprises all the elements of the configuration shown on FIG. 49B. The elements of the breathing system 200M3 are mounted on the automobile frame, and the breathing system 200M3 supplies the breathable gases to the automobile driver and passengers. The controller 210M3 can be integrated into the automobile's ventilation and air conditioning system controller. The breathable gases can be supplied to a breathing zone, which means the breathable gases are released into an automobile's cabin at a specified location, for example, near by a driver's seat, or near by a select passenger's seat, or near by the both of them. The direction of the flow of the released breathable gases can be also adjusted so that the breathable gases are supplied to by inhaling organs as directly as possible. The measures mentioned above can be integrated with the automobile's ventilation and air conditioning system.

FIG. 52A illustrates a breathing system 200M3 configured for use with a residential compartment. The breathing system 200M3 comprises all the elements of the configuration shown on FIG. 49A. The breathing system 200M3 can be installed at any convenient location. The controller 210M3 can be integrated into the compartment's ventilation and air conditioning system controller. The breathable gases can be supplied to a breathing zone, which means the breathable gases are released into a residential compartment at a specified location, for example, near by a group of people, or inside a select room. The direction of the flow of the released breathable gases can be also adjusted so that the breathable gases are supplied to by inhaling organs as directly as possible. The measures mentioned above can be integrated with the compartment's ventilation and air conditioning system.

FIG. 52B illustrates a breathing system 200M3 configured for use with a residential compartment. The breathing system 200M3 comprises all the elements of the configuration shown on FIG. 49B. The breathing system 200M3 can be installed at any convenient location. The controller 210M3 can be integrated into the compartment's ventilation and air conditioning system controller. The breathable gases can be supplied to a breathing zone, which means the breathable gases are released into a residential compartment at a specified location, for example, near by a group of people, or inside a select room. The direction of the flow of the released breathable gases can be also adjusted so that the breathable gases are supplied to by inhaling organs as directly as possible. The measures mentioned above can be integrated with the compartment's ventilation and air conditioning system.

FIG. 53A illustrates another example of a breathing system 200M3 configured for use with a residential compartment. The breathing system 200M3 comprises all the elements of the configuration shown on FIG. 52A, wherein the container of liquefied oxygen 501A and the container of liquefied inert gas 501B are mounted on a mobile platform 118, so that the container of liquefied oxygen 501A and the container of liquefied inert gas 501B can easily be moved to a dispenser of liquefied breathable gases 401 for dispensing purposes, and back to the compartment when the container of liquefied oxygen 501A and the container of liquefied inert gas 501B are replenished with the amount of the liquefied breathable gases corresponding to their capacity.

FIG. 53B illustrates another example of a breathing system 200M3 configured for use with a residential compartment. The breathing system 200M3 comprises all the elements of the configuration shown on FIG. 52B, wherein the container of liquefied breathable gases 501 is mounted on a mobile platform 118, so that the container of liquefied breathable gases 501 can easily be moved to a dispenser of liquefied breathable gases 401 for dispensing purposes, and back to the compartment when the container of liquefied breathable gases 501 is replenished with the amount of the liquefied breathable gases corresponding to its capacity.

### BRIEF DESCRIPTION OF DRAWINGS

FIG. 1A illustrates a system configured to supply breathable gases to civilian people at an appropriate proportion.

FIG. 1B illustrates an arrangement of a system configured to supply breathable gases to civilian people.

FIG. 1C illustrates an arrangement of a system configured to supply breathable gases to civilian people.

FIG. 1D illustrates an arrangement of a system configured to supply breathable gases to civilian people.

FIG. 1E illustrates an arrangement of a system configured to supply breathable gases to civilian people.

FIG. 1F illustrates an arrangement of a source of breathable gases.

FIG. 1G illustrates an arrangement of a source of breathable gases.

FIG. 2 illustrates a dispenser of breathable gases configured to receive breathable gases from a source of breathable gases, dispense the breathable gases to a container of breathable gases in response to a corresponding signal from a communication unit, and control a pressure of the dispensed breathable gases.

FIG. 3 illustrates a dispenser of breathable gases configured to receive breathable gases from a source of breathable gases, dispense the breathable gases to a container of breathable gases in response to a corresponding signal from a communication unit, control the pressure of the dispensed breathable gases, and measure the temperature of the dispensed breathable gases.

FIG. 4 illustrates a distributor of breathable gases configured to receive breathable gases from a source of breathable gases and conduct the breathable gases to at least one inhalation unit in response to a corresponding signal from a communication unit.

FIG. 5 illustrates an example of a communication unit configured to receive an input from a user in a form of a payment card payment, communicate the input to a controller, and communicate an information of interest from the controller to the user.

FIG. 6 illustrates an example of a communication unit configured to receive an input from a user in a form of a scan-able bar-code, communicate the input to a controller, and communicate an information of interest from the controller to the user.

FIG. 7 illustrates an example of a communication unit configured to receive an input from a user in a form of a scan-able bar-code, receive an input from the user in a form of a payment card payment, communicate the input to a controller, and communicate an information of interest from the controller to the user.

FIG. 8A illustrates a connected state of the dispenser of breathable gases and the container of breathable gases.

FIG. 8B illustrates a disconnected state of the dispenser of breathable gases and the container of breathable gases.

FIG. 9 illustrates a breathing system configured to store a reasonable amount of breathable gases in a portable source of breathable gases and to conduct the breathable gases from the portable source of breathable gases to an inhalation unit in response to an input from a user.

FIG. 10A illustrates a breathing system configured for use with a bicycle.

FIG. 10B illustrates a breathing system configured for use with a motorized vehicle.

FIG. 11 illustrates a breathing system configured for use with an automobile.

FIG. 12 illustrates a breathing system configured for use within a residential compartment.

FIG. 13 illustrates an example of a breathing system configured for use within a residential compartment.

FIG. 14 illustrates an arrangement of a source of breathable gases.

FIG. 15 illustrates a storage of breathable gases.

FIG. 16 illustrates an arrangement of a source of breathable gases.

FIG. 17 illustrates a mobile container of breathable gases.

FIG. 18 illustrates an arrangement of a source of breathable gases.

FIG. 19 illustrates an arrangement of a source of breathable gases.

FIG. 20 illustrates an arrangement of a liquid-to-gas converter configured to receive liquefied oxygen and liquefied inert gas from a source of liquefied breathable gases, evaporate the received liquefied oxygen, evaporate the received liquefied inert gas, mix the evaporated oxygen and the evaporated inert gas in an appropriate proportion, and supply the mixed breathable gases to a breathable gases consumer.

FIG. 21 illustrates an arrangement of a liquid-to-gas converter configured to receive liquefied oxygen and liquefied inert gas from a source of liquefied breathable gases, evaporate the received liquefied oxygen, evaporate the received liquefied inert gas, mix the evaporated oxygen and the evaporated inert gas in an appropriate proportion, and supply the mixed breathable gases to a breathable gases consumer.

FIG. 22 illustrates an arrangement of a liquid-to-gas converter configured to receive liquefied oxygen and liquefied inert gas from a source of liquefied breathable gases, mix the liquefied oxygen and the liquefied inert gas in an appropriate proportion, evaporate the mixture of the liquefied oxygen and the liquefied inert gas, and supply the evaporated breathable gases to a breathable gases consumer.

FIG. 23 illustrates an arrangement of a liquid-to-gas converter configured to receive liquefied oxygen and liquefied inert gas from a source of liquefied breathable gases, mix the liquefied oxygen and the liquefied inert gas in an appropriate proportion, evaporate the mixture of the liquefied oxygen and the liquefied inert gas, and supply the evaporated breathable gases to a breathable gases consumer.

FIG. 24 illustrates an arrangement of a source of breathable gases.

FIG. 25 illustrates an arrangement of a source of breathable gases.

FIG. 26 illustrates an arrangement of a source of breathable gases.

FIG. 27 illustrates an arrangement of a source of breathable gases.

FIG. 28 illustrates an arrangement of a source of breathable gases.

FIG. 29 illustrates an arrangement of a source of breathable gases.

FIG. 30 illustrates an arrangement of a source of breathable gases.

FIG. 31 illustrates an arrangement of a source of breathable gases.

FIG. 32 illustrates an arrangement of a source of breathable gases.

FIG. 33 illustrates an arrangement of a source of breathable gases.

FIG. 34 illustrates an arrangement of a source of breathable gases.

FIG. 35 illustrates an arrangement of a source of breathable gases.

FIG. 36 illustrates an arrangement of a source of breathable gases.

FIG. 37 illustrates an arrangement of a source of breathable gases.

FIG. 38 illustrates an arrangement of a source of breathable gases.

FIG. 39 illustrates a dispenser of liquefied breathable gases configured to receive liquefied breathable gases from a source of liquefied breathable gases, dispense the liquefied oxygen to a container of liquefied oxygen, and liquefied inert gas to a container of liquefied inert gas, in a response to an input from a user, and control the flow rate of dispensed liquefied breathable gases.

FIG. 40A illustrates a connected state of the dispenser of liquefied breathable gases, a container of liquefied oxygen and a container of liquefied inert gas.

FIG. 40B illustrates a disconnected state of the dispenser of liquefied breathable gases, a container of liquefied oxygen and a container of liquefied inert gas.

FIG. 41 illustrates an arrangement of a source of liquefied breathable gases.

FIG. 42 illustrates a storage of liquefied oxygen and a storage of liquefied inert gas.

FIG. 43 illustrates an arrangement of a source of liquefied breathable gases.

FIG. 44 illustrates a mobile container of liquefied oxygen and a mobile container of liquefied inert gas.

FIG. 45 illustrates an arrangement of a source of liquefied breathable gases.

FIG. 46 illustrates an arrangement of a source of liquefied breathable gases.

FIG. 47 illustrates an arrangement of a source of liquefied breathable gases.

FIG. 48 illustrates an arrangement of a source of liquefied breathable gases.

FIG. 49 illustrates a breathing system configured to store a reasonable amount of liquefied breathable gases in a portable source of liquefied breathable gases, evaporate the liquefied breathable gases, and conduct the evaporated breathable gases to an inhalation unit in response to an input from a user.

FIG. 50A illustrates breathing system configured for use with a bicycle.

FIG. 50B illustrates a breathing system configured for use with a motorized vehicle.

FIG. 51 illustrates a breathing system configured for use with an automobile.

FIG. 52 illustrates a breathing system configured for use with a residential compartment.

FIG. 53 illustrates an example of a breathing system configured for use with a residential compartment.

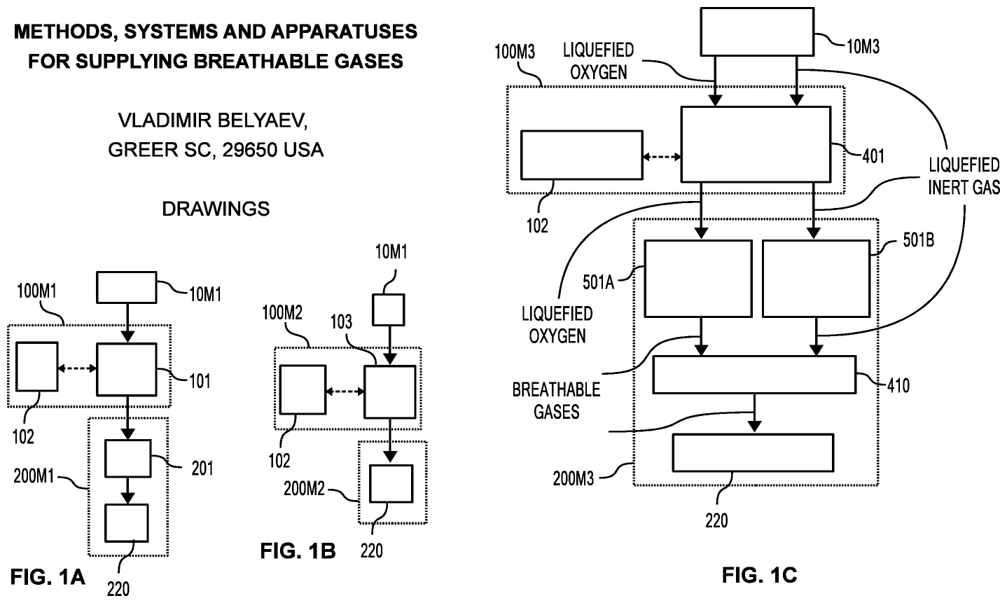

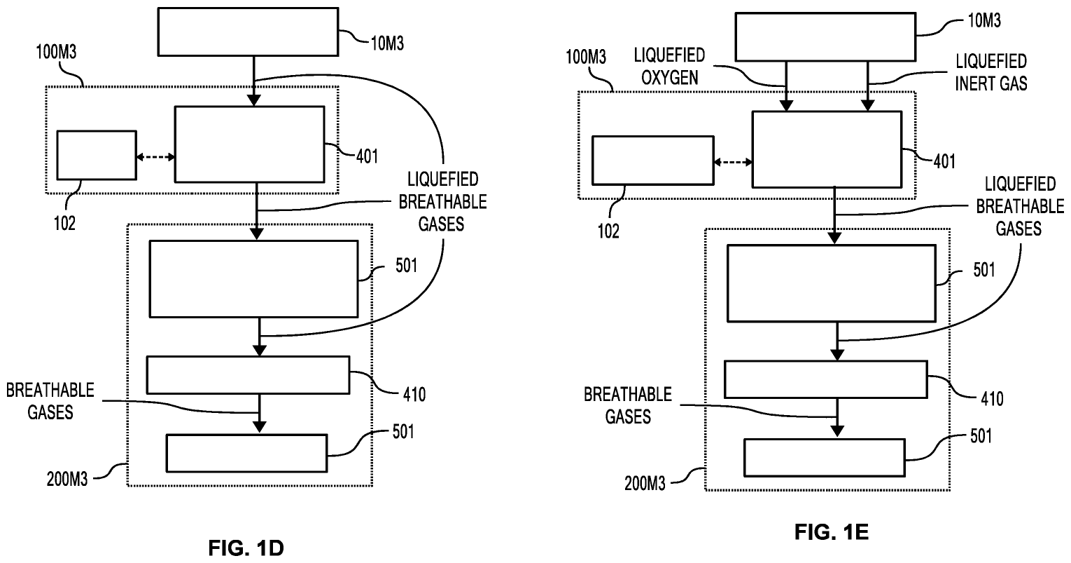

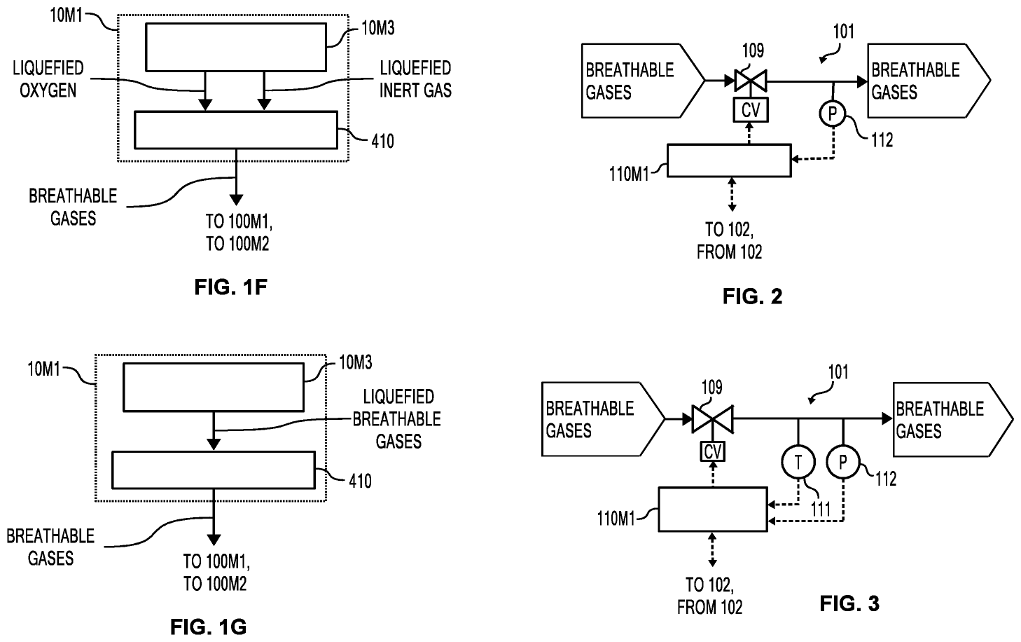

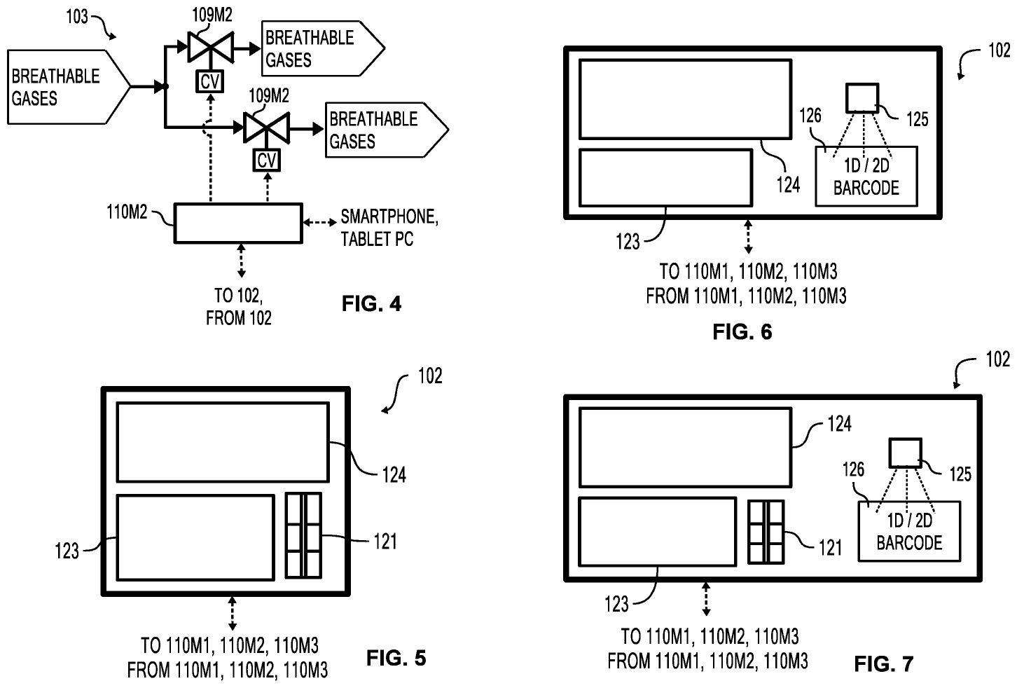

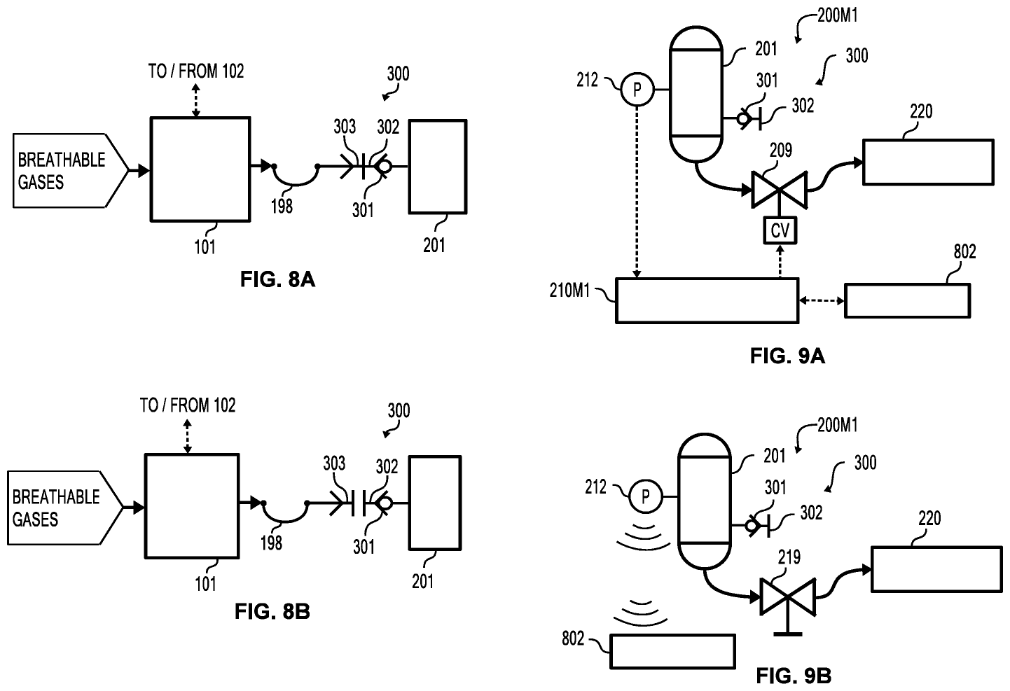

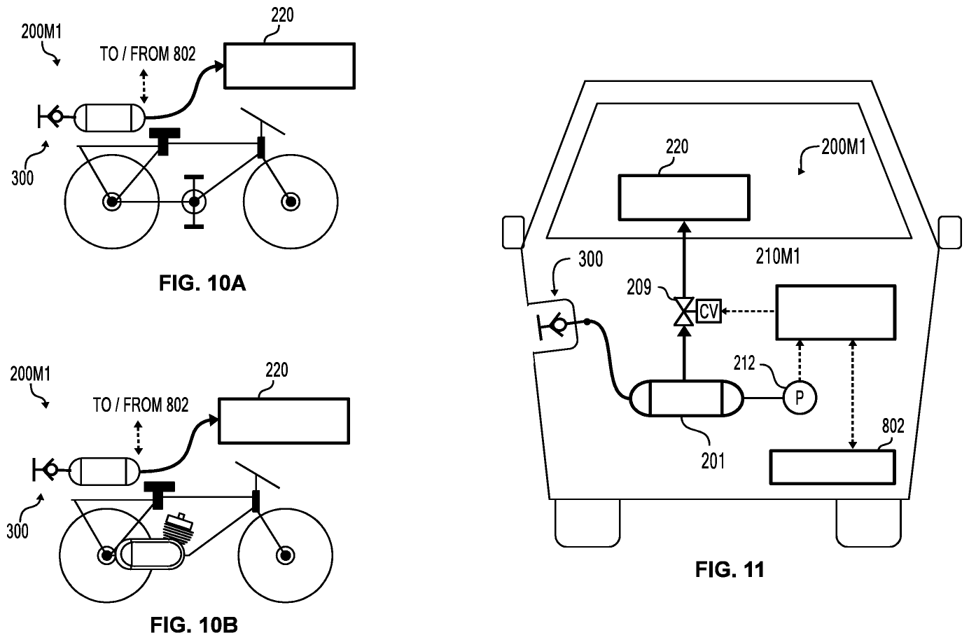

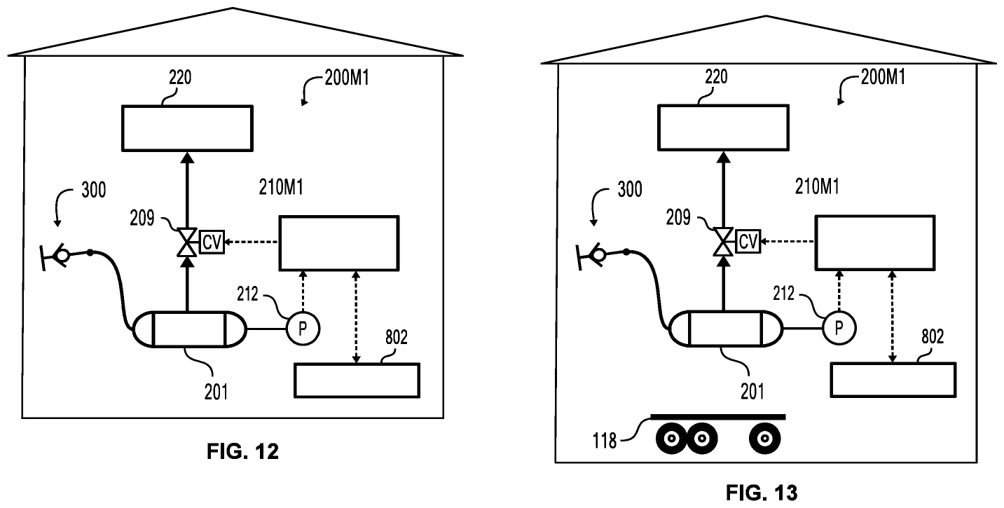

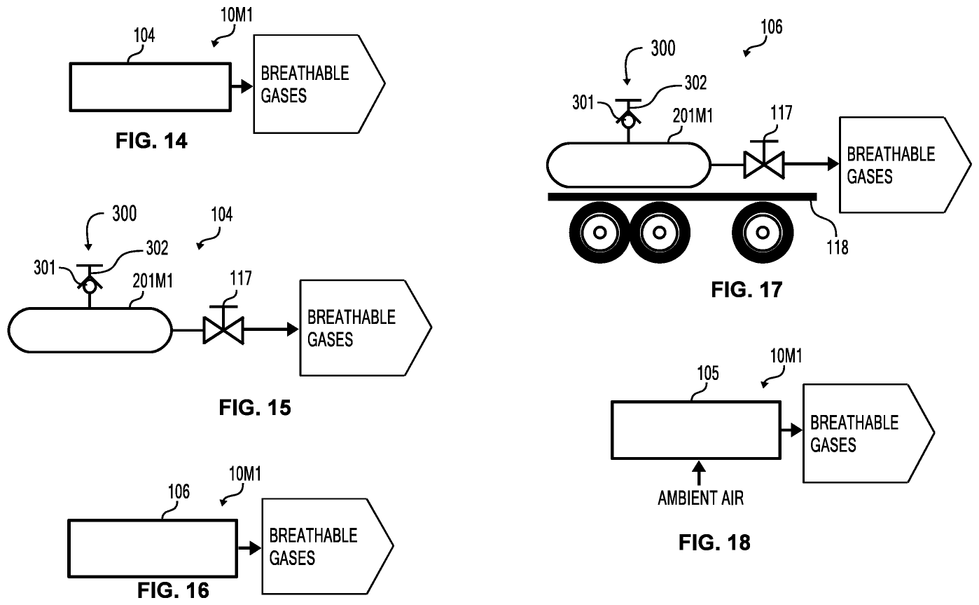

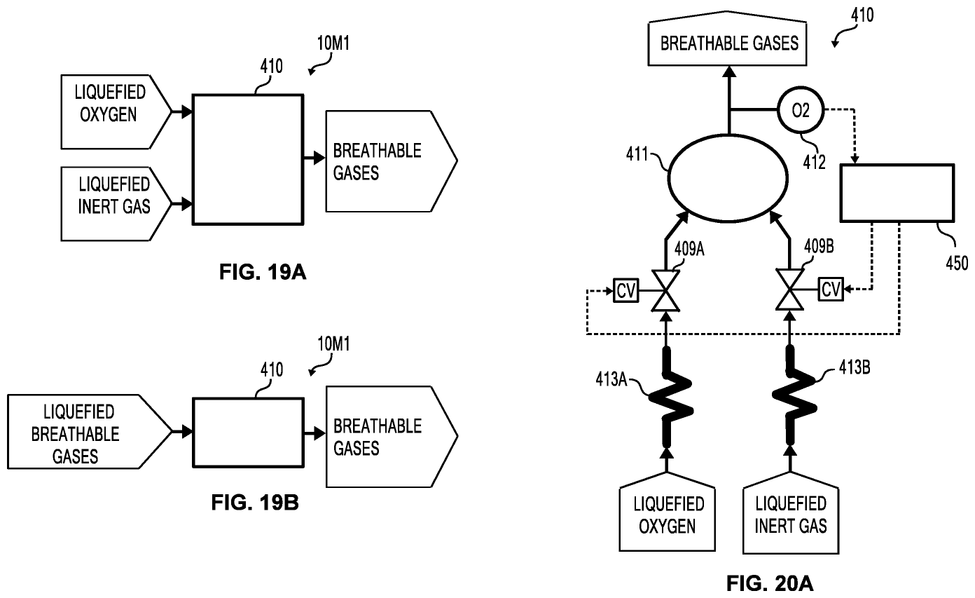

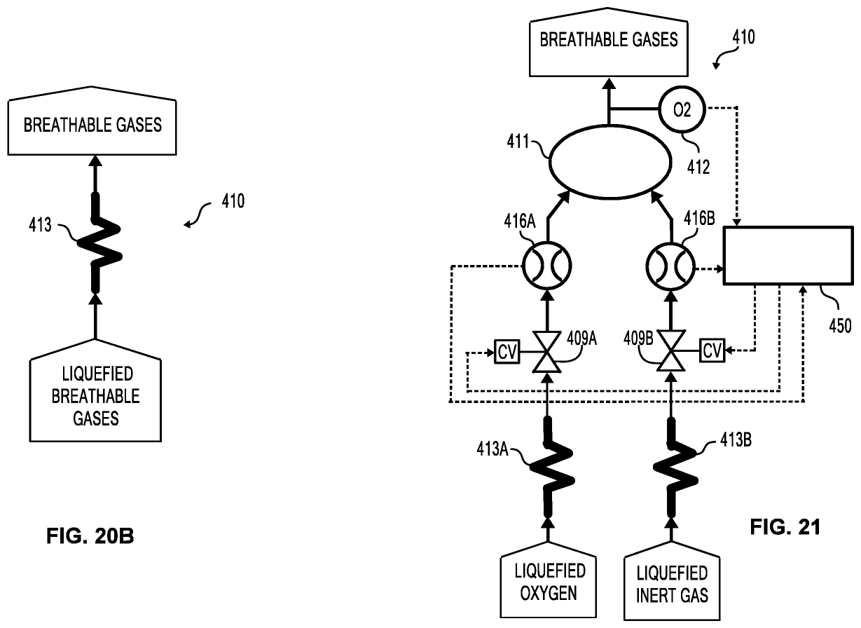

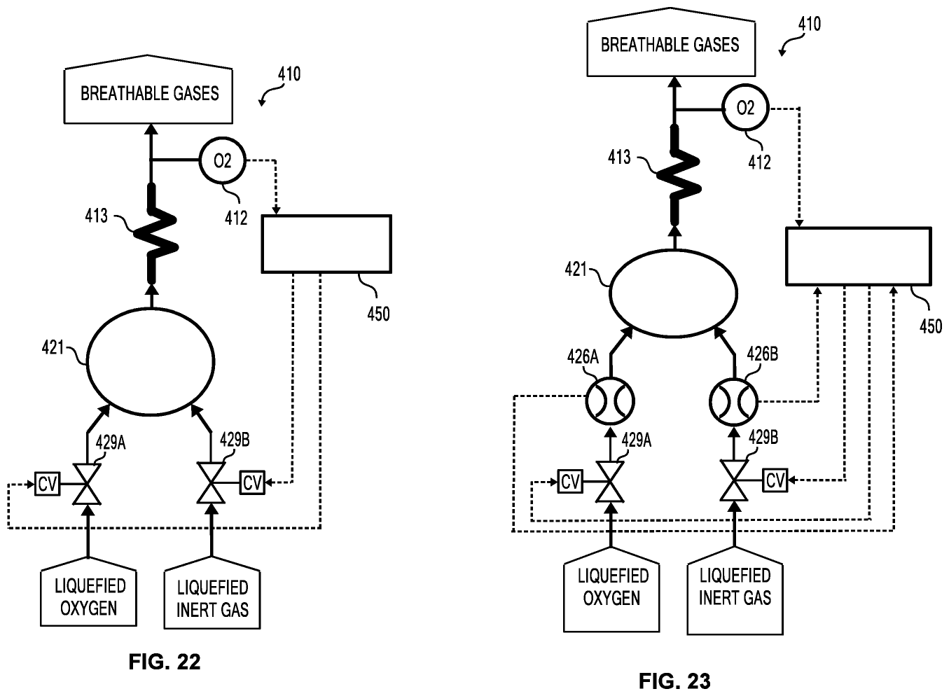

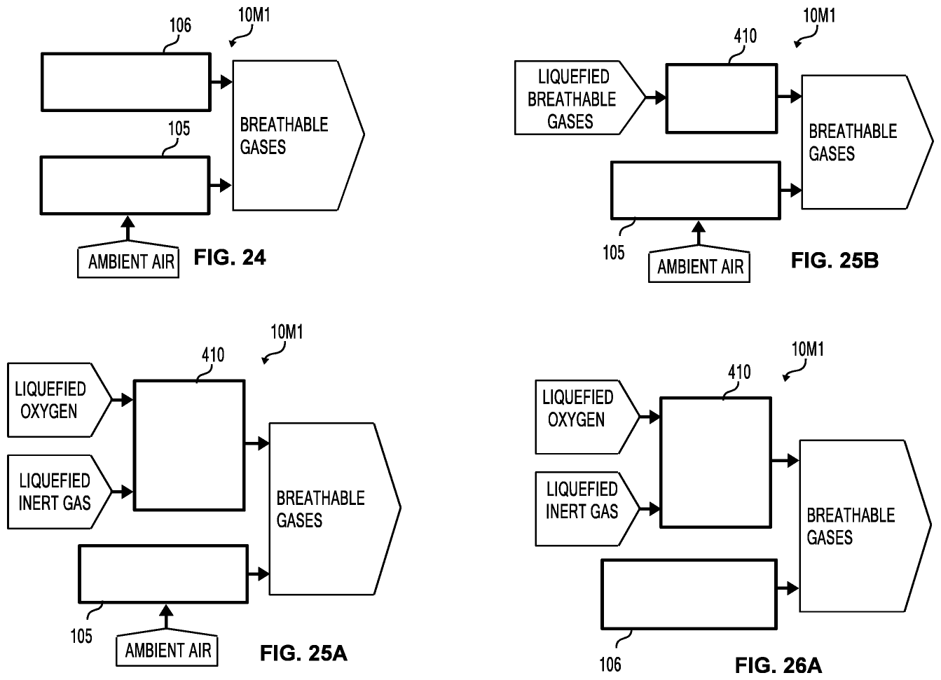

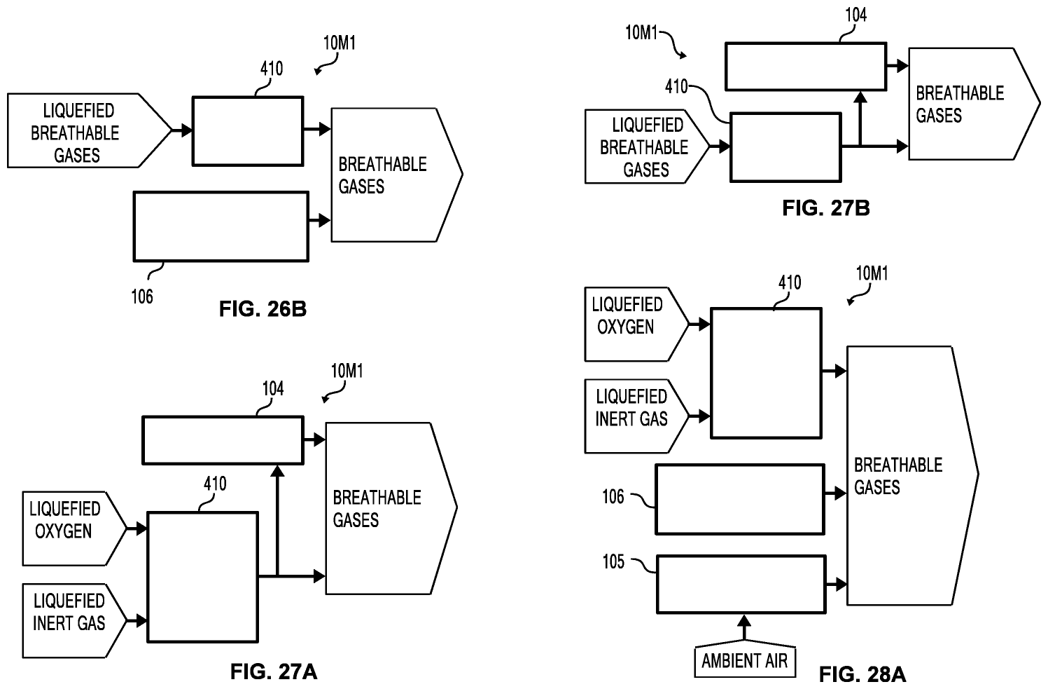

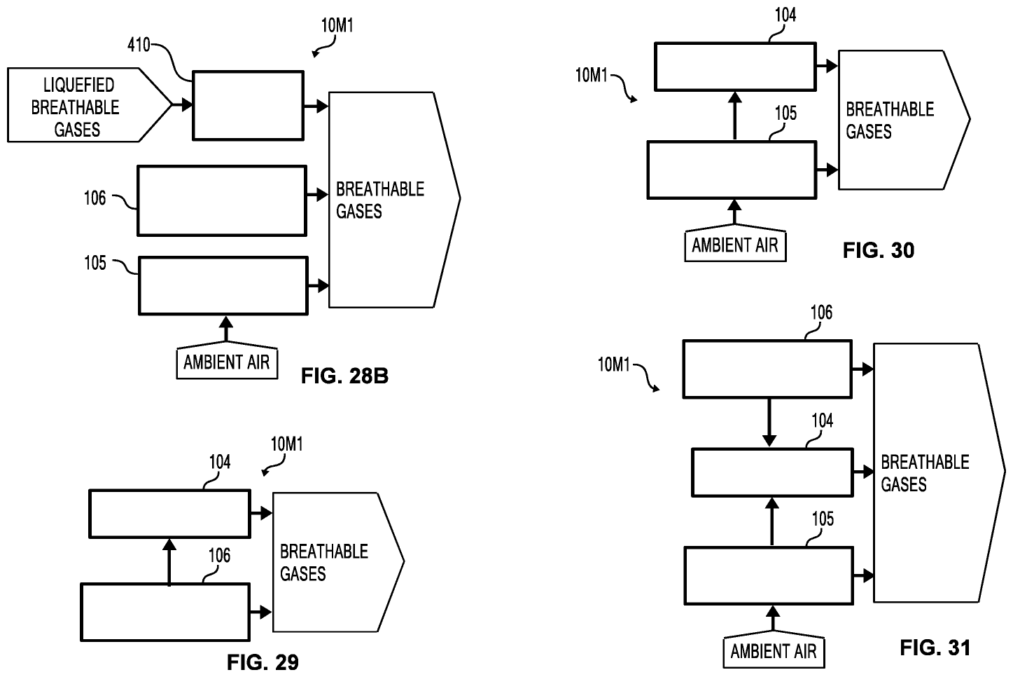

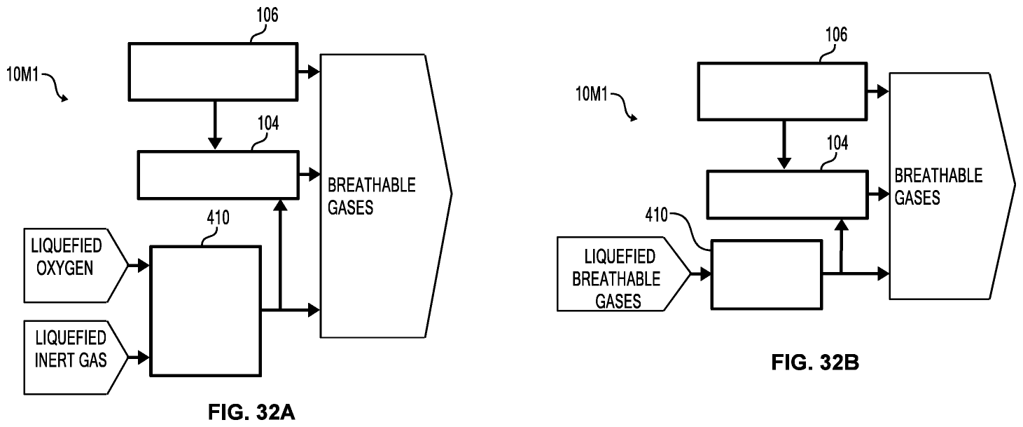

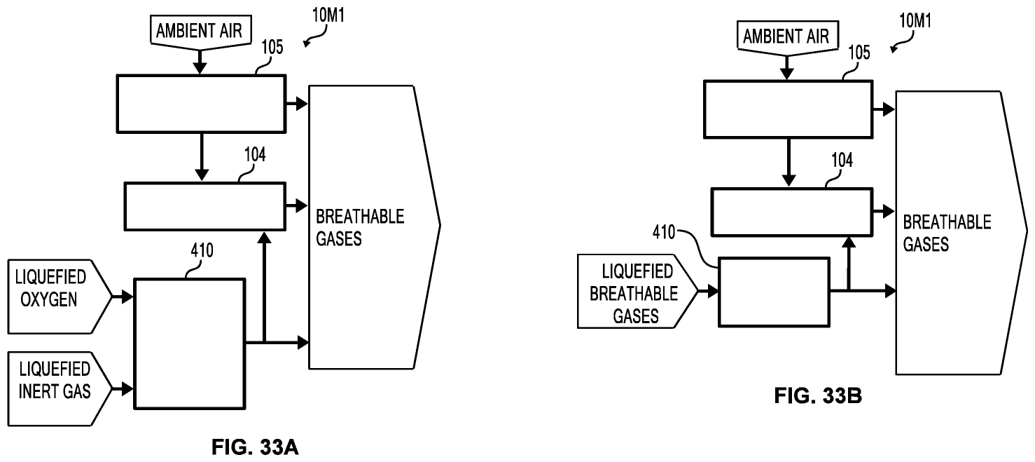

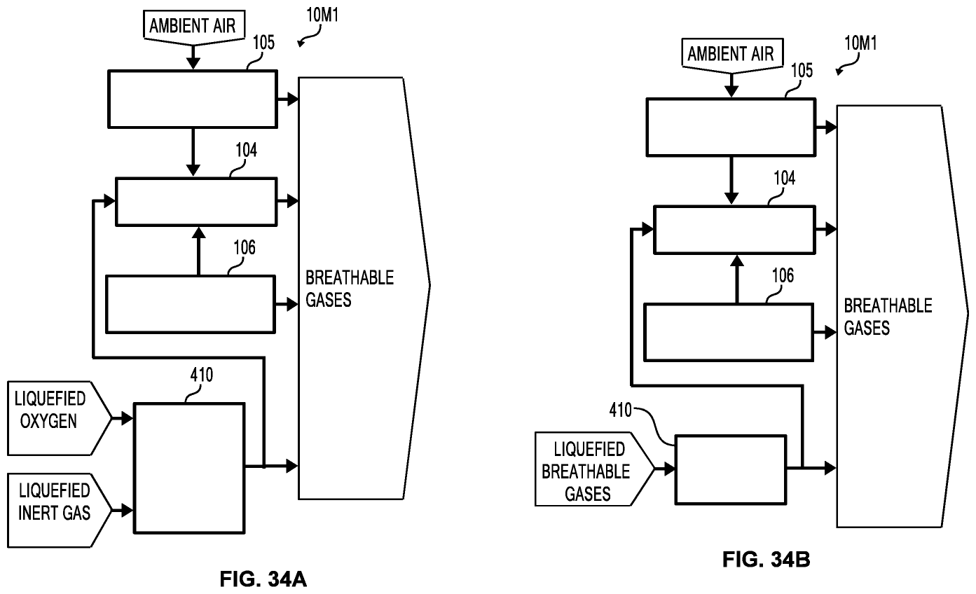

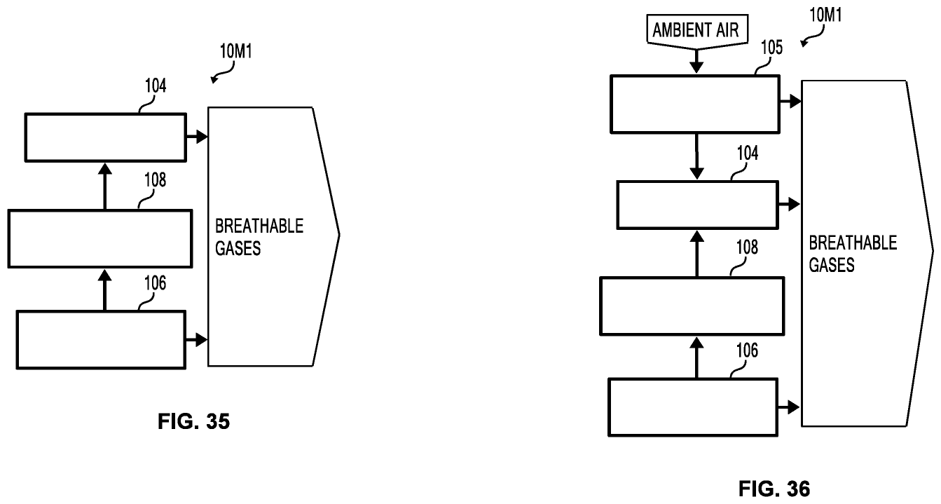

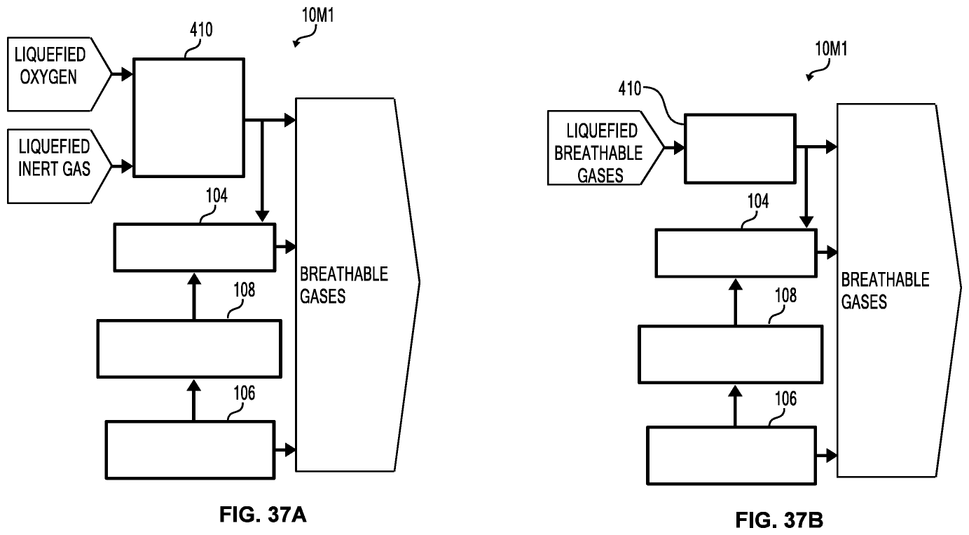

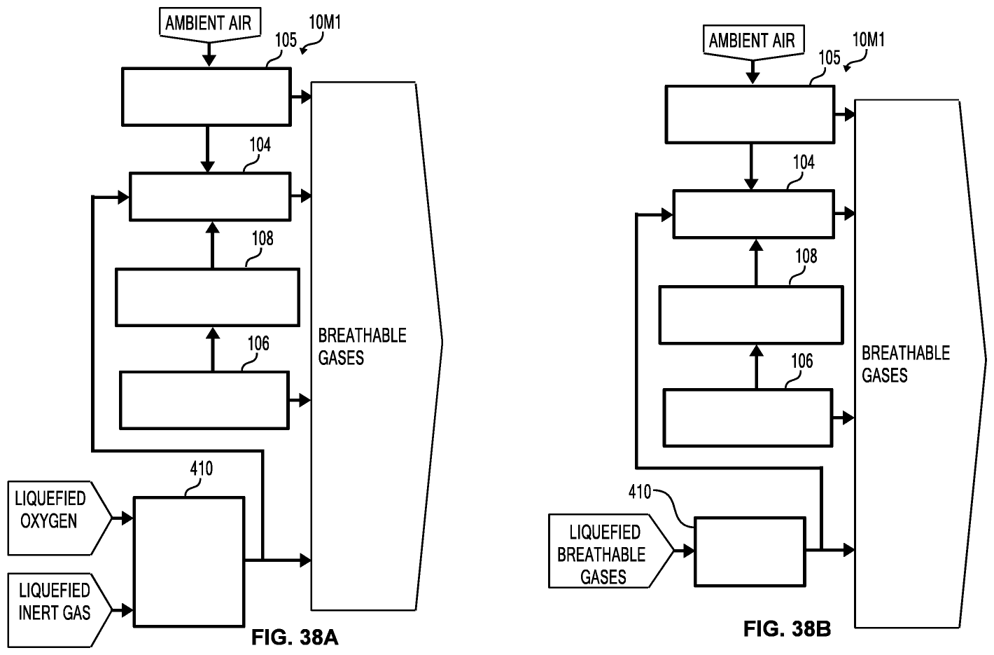

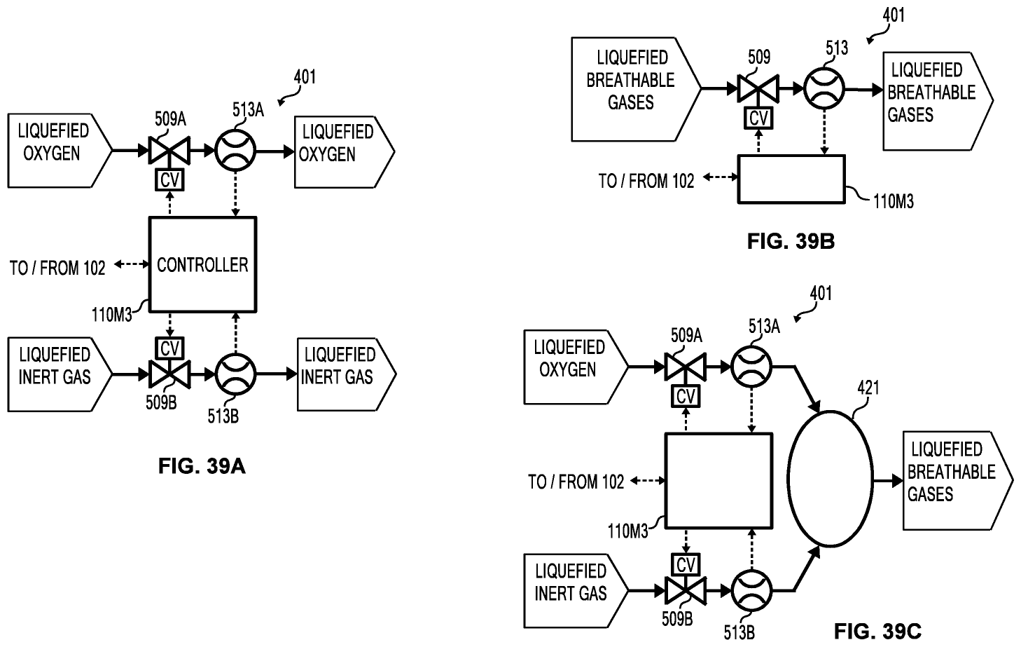

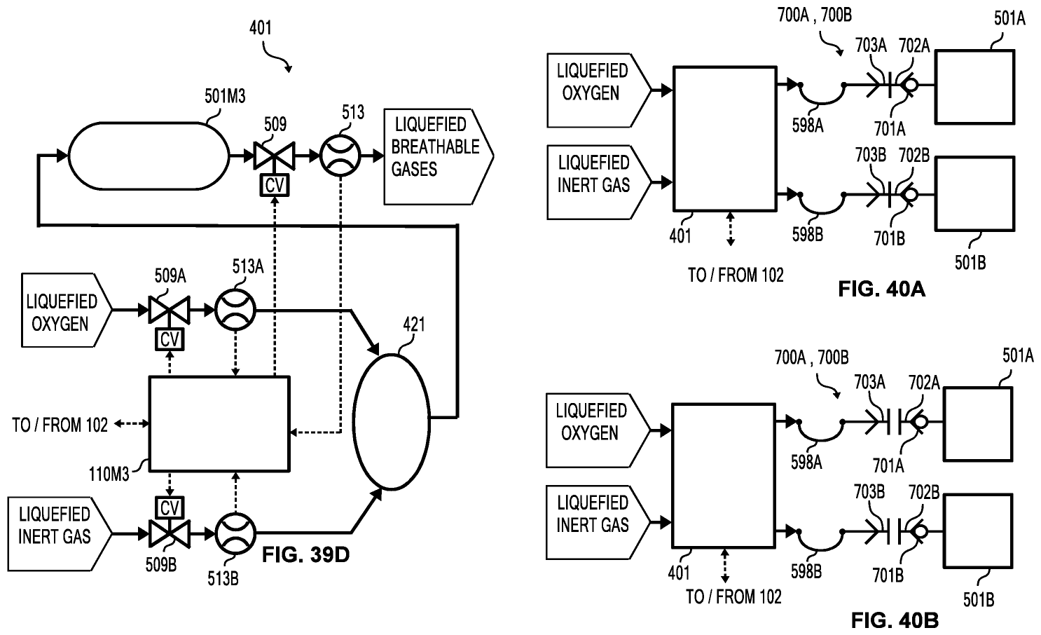

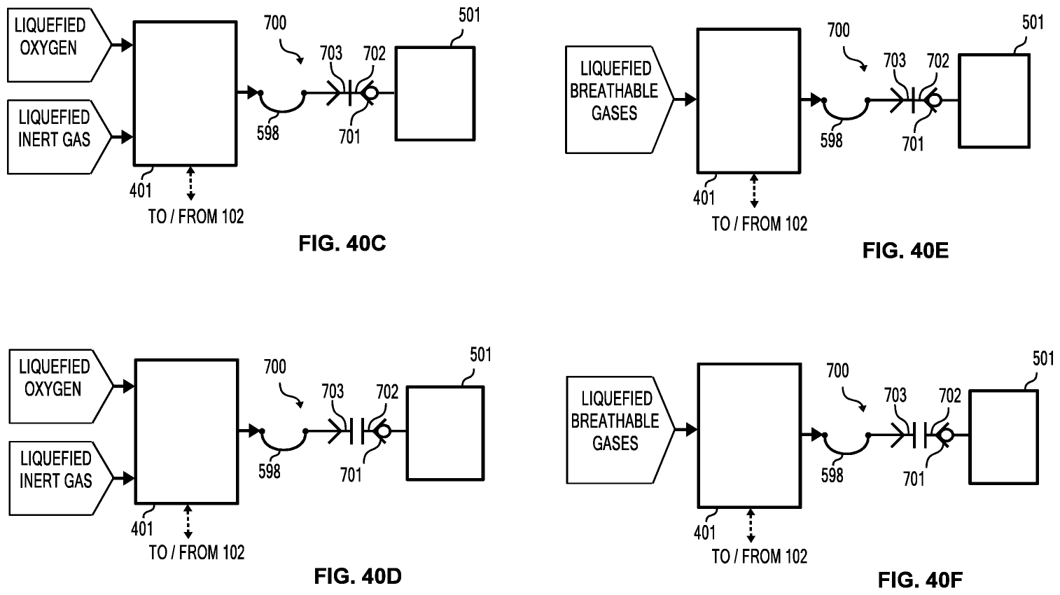

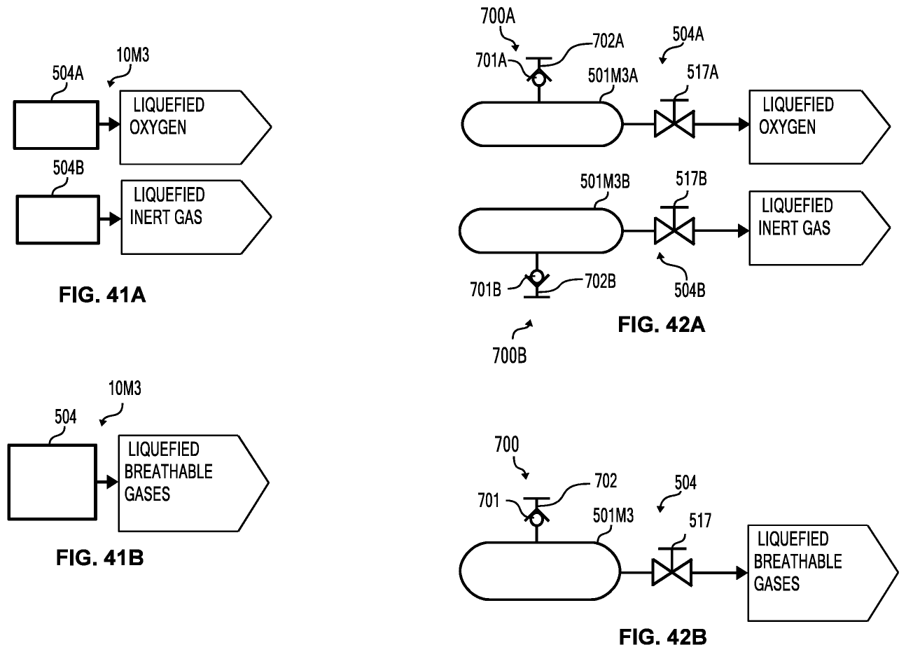

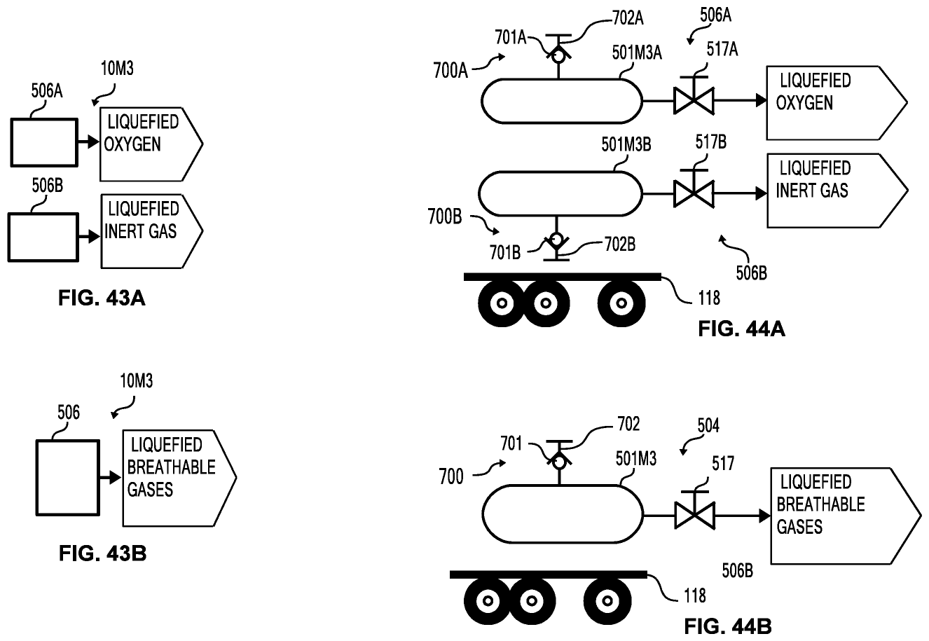

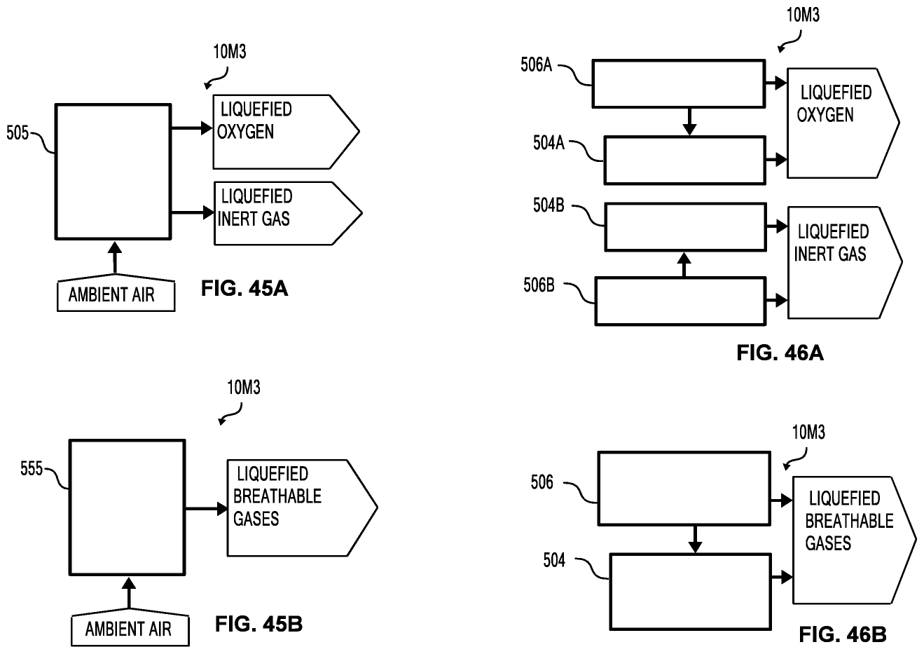

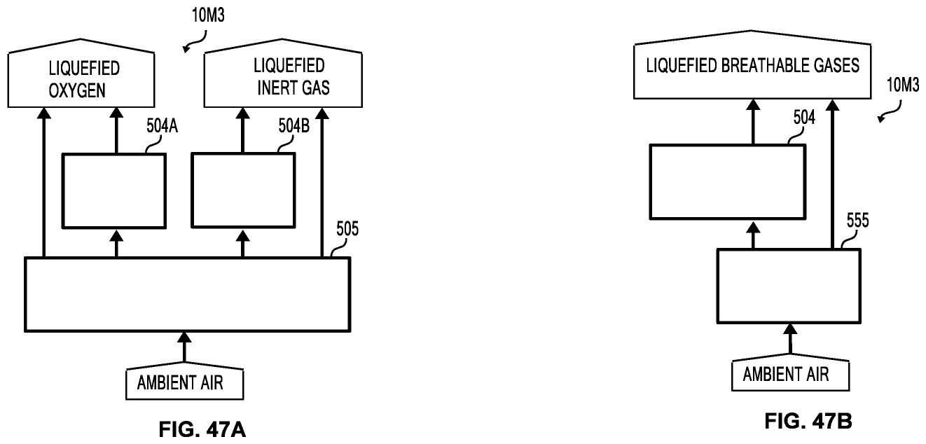

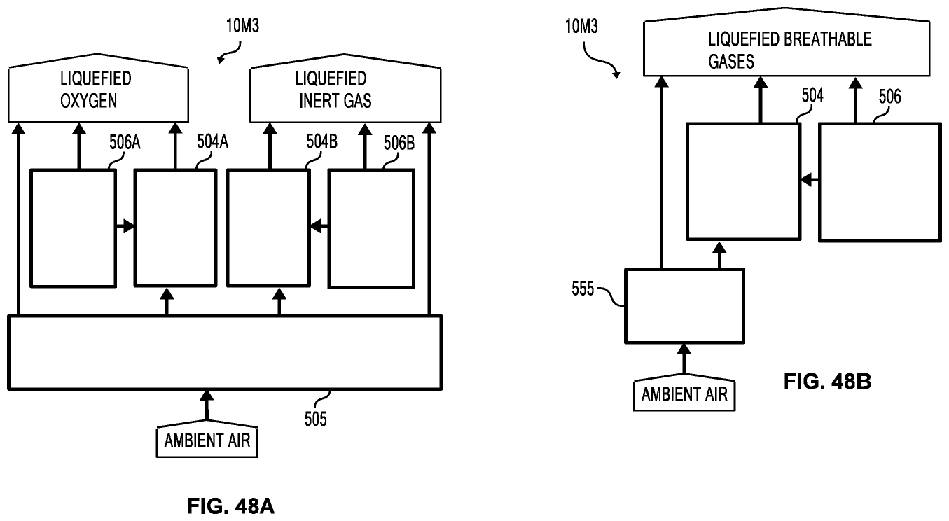

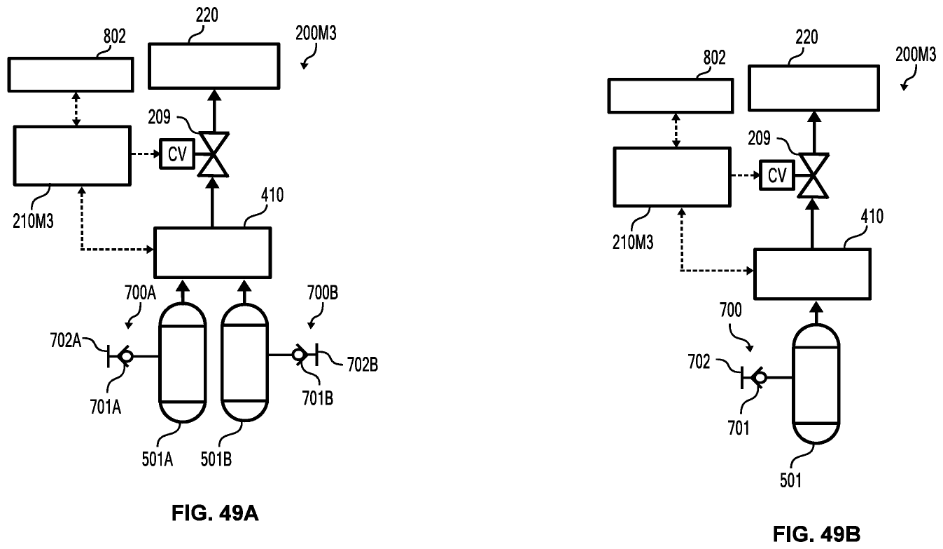

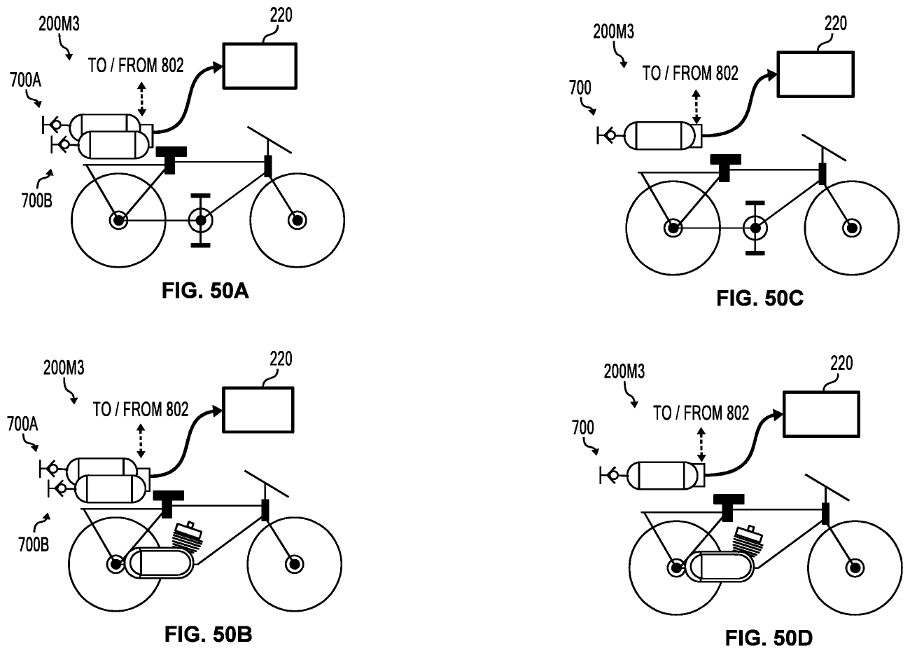

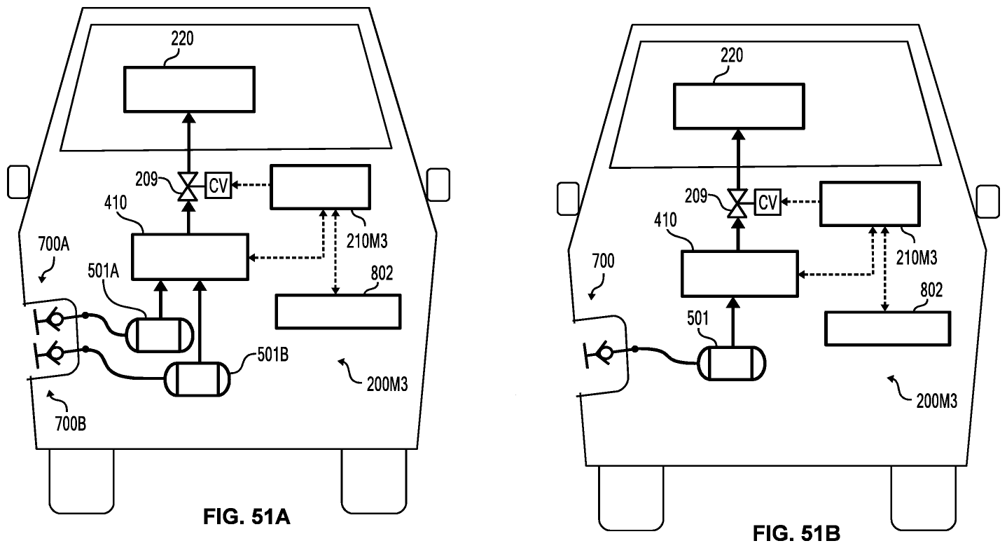

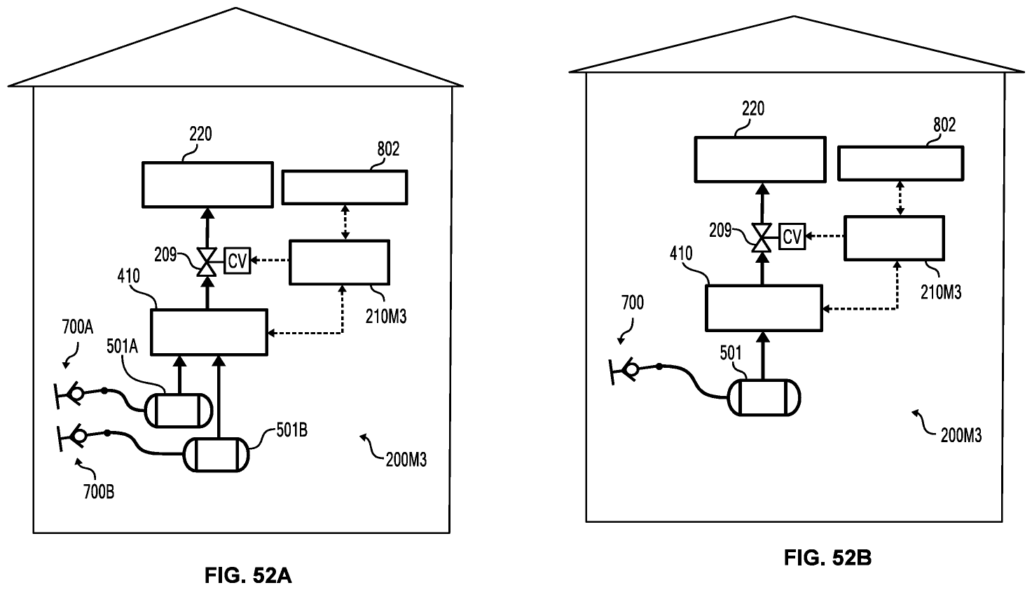

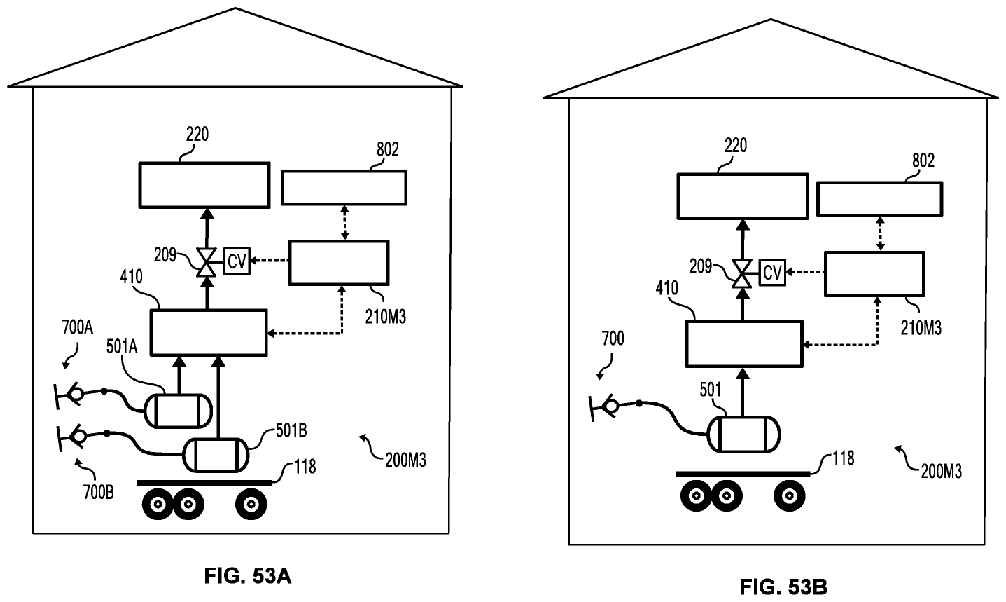

### BEST MODE FOR CARRYING OUT THE INVENTION

A best mode for carrying out the invention is disclosed in the description. A variety of disclosed arrangements indicate flexibility of the invention in terms of efficiency and industrial applicability, as well as protects the invention from a use of a relatively less efficient arrangements.

### INDUSTRIAL APPLICABILITY

As described in Background Art section, the present invention can be manufactured and deployed utilizing SCUBA and SCBA, as well as CNG and LNG, manufacturing and deployment technologies and strategies.

1. A compressed breathable gases dispensing system comprising:
a source of breathable gases configured to store and supply a reasonable amount of breathable gases; and
a dispenser of compressed breathable gases in communication with the source of breathable gases and dispensing compressed breathable gases into one or more containers of compressed breathable gases;
wherein the dispenser of compressed breathable gases comprises a communication unit in a form of preferably but not limited to a payment receiving terminal, the communication unit is configured to receive payment transactions for the dispensed compressed breathable gases and communicate activating signal to the dispenser of compressed breathable gases to dispense compressed breathable gases to a container of compressed breathable gases.

2. A breathable gases distributing system comprising:
a source of breathable gases configured to contain and supply a reasonable amount of breathable gases;
a breathable gases distributor in communication with the source of breathable gases and distributing breathable gases from the source of breathable gases to one or more inhalation units;
wherein the distributor of breathable gases comprises a communication unit in a form of preferably but not limited to a payment receiving terminal, the communication unit is configured to receive payment transactions for the distributed compressed breathable gases and communicate activating signal to the distributor of breathable gases to distribute breathable gases to one or more inhalation units for a paid period of time.

3. The system of claim 1 and claim 2, wherein the source of breathable gases comprises a storage of breathable gases.

4. The system of claim 1 and claim 2, wherein the source of breathable gases comprises a mobile container of breathable gases.

5. The system of claim 1 and claim 2, wherein the source of breathable gases comprises a compressor of breathable gases.

6. The system of claim 1 and claim 2, wherein the source of breathable gases comprises a combination of a storage of breathable gases and a mobile container of breathable gases.

7. The system of claim 1 and claim 2, wherein the source of breathable gases comprises a combination of a compressor of breathable gases and a mobile container of breathable gases.

8. The system of claim 1 and claim 2, wherein the source of breathable gases comprises a combination of a storage of breathable gases and a compressor of breathable gases.

9. The system of claim 1 and claim 2, wherein the source of breathable gases comprises a combination of a storage of breathable gases, a mobile container of breathable gases, and a compressor of breathable gases.

10. The system of claim 6 and claim 7, wherein the source of breathable gases further comprises a trans-compressor in communication with the mobile container of breathable gases and in communication with the storage of breathable gases and conducting breathable gases from the mobile container of breathable gases to the storage of breathable gases at a higher pressure than the current pressure in the mobile container of breathable gases, so that to maintain an appropriate pressure level in the storage of breathable gases.

11. The system of claims 3, 4, 5, 6, 7, 8, 9 and 10, wherein the source of breathable gases further comprises:
a source of liquefied breathable gases; and
a liquid-to-gas converter in communication with the source of liquefied breathable gases and evaporating liquefied breathable gases.

12. The system of claim 1 and claim 2, wherein the source of breathable gases comprises:
a source of liquefied breathable gases; and
a liquid-to-gas converter in communication with the source of liquefied breathable gases and evaporating liquefied breathable gases.

13. The system of claim 11 and claim 12, wherein the liquid-to-gas converter receiving liquefied oxygen and liquefied inert gas separately from the source of liquefied breathable gases, evaporating them separately, and mixing them in a proportion so that to produce a mixture of a composition appropriate for breathing.

14. The system of claim 11 and claim 12, wherein the liquid-to-gas converter receiving liquefied oxygen and liquefied inert gas separately from the source of liquefied breathable gases, mixing them in a proportion so that to produce a mixture of a composition appropriate for breathing, and evaporating the mixture to produce gaseous breathable gases.

15. The system of claim 11 and claim 12, wherein the source of liquefied breathable gases comprises a storage of liquefied breathable gases.

16. The system of claim 11 and claim 12, wherein the source of liquefied breathable gases comprises a mobile container of liquefied breathable gases.

17. The system of claim 11 and claim 12, wherein the source of liquefied breathable gases comprises an air liquefaction system.

18. The system of claim 11 and claim 12, wherein the source of liquefied breathable gases comprises a combination of an air liquefaction system and a storage of liquefied breathable gases.

19. The system of claim 11 and claim 12, wherein the source of liquefied breathable gases comprises a combination of a mobile container of liquefied breathable gases and a storage of liquefied breathable gases.

20. The system of claim 11 and claim 12, wherein the source of liquefied breathable gases comprises a combination of a mobile container of liquefied breathable gases and an air liquefaction system.

21. The system of claim 11 and claim 12, wherein the source of liquefied breathable gases comprises a combination of a storage of liquefied breathable gases, a mobile container of liquefied breathable gases and an air liquefaction system.

22. The system of claim 11 and claim 12, wherein the source of liquefied breathable gases comprises a storage of liquefied oxygen and liquefied inert gas.

23. The system of claim 11 and claim 12, wherein the source of liquefied breathable gases comprises a mobile container of liquefied oxygen and liquefied inert gas.

24. The system of claim 11 and claim 12, wherein the source of liquefied breathable gases comprises an air separation and liquefaction system.

25. The system of claim 11 and claim 12, wherein the source of liquefied breathable gases comprises a combination of an air separation and liquefaction system and a storage of oxygen and liquefied inert gas.

26. The system of claim 11 and claim 12, wherein the source of liquefied breathable gases comprises a combination of a mobile container of liquefied oxygen and liquefied inert gas and a storage of liquefied oxygen and liquefied inert gas.

27. The system of claim 11 and claim 12, wherein the source of liquefied breathable gases comprises a combination of a mobile container of liquefied oxygen and liquefied inert gas and an air separation and liquefaction system.

28. The system of claim 11 and claim 12, wherein the source of liquefied breathable gases comprises a combination of a storage of liquefied oxygen and liquefied inert gas, a mobile container of liquefied oxygen and liquefied inert gas and an air separation and liquefaction system.

29. A liquefied breathable gases dispensing system comprising:
a source of breathable gases configured to store and supply a reasonable amount of liquefied breathable gases; and
a dispenser of liquefied breathable gases in communication with the source of breathable gases and dispensing liquefied breathable gases into one or more containers of liquefied breathable gases;
wherein the dispenser of liquefied breathable gases comprises a communication unit in a form of preferably but not limited to a payment receiving terminal, the communication unit is configured to receive payment transactions for the dispensed liquefied breathable gases and communicate activating signal to the dispenser of liquefied breathable gases to dispense liquefied breathable gases to one or more containers of liquefied breathable gases.

30. A breathing apparatus, comprising:
a container of breathable gases storing and supplying a reasonable amount of compressed breathable gases;
a dispensing connection with a one-way check valve in communication with the container of compressed breathable gases and allowing to dispense compressed breathable gases to the container of compressed breathable gases;
an inhalation unit in communication with the container of compressed breathable gases and releasing breathable gases around inhalation organs of a user;
a controlled valve disposed between outlet of the container of compressed breathable gases and the inhalation unit and opening and closing the flow of breathable gases from the container of compressed breathable gases to the inhalation unit;
a controller in communication with the controlled valve and transmitting activating signals to the controlled valve to open and close the flow of breathable gases;
a communicator in communication with the controller and receiving an input from a user and transmitting corresponding signal to the controller to open and to close the flow of breathable gases.

31. A breathing apparatus, comprising:
a container of liquefied breathable gases storing and supplying a reasonable amount of liquefied breathable gases;
a dispensing connection with a one-way check valve in communication with the container of liquefied breathable gases and allowing to dispense liquefied breathable gases to the container of liquefied breathable gases;
a liquid-to-gas converter in communication with the container of liquefied breathable gases and evaporating liquefied breathable gases;
an inhalation unit in communication with the liquid-to-gas converter and releasing breathable gases around inhalation organs of a user;
a controlled valve disposed between outlet of the liquid-to-gas converter and the inhalation unit and opening and closing the flow of breathable gases from the liquid-to-gas converter to the inhalation unit;
a controller in communication with the controlled valve and transmitting activating signals to the controlled valve to open and close the flow of breathable gases;
a communicator in communication with the controller and receiving an input from a user and transmitting corresponding signal to the controller to open and to close the flow of breathable gases.

32. A breathing apparatus, comprising:
a container of liquefied oxygen storing and supplying a reasonable amount of liquefied oxygen;
a container of liquefied inert gas storing and supplying a reasonable amount of liquefied inert gas;
a dispensing connection with a one-way check valve in communication with the container of liquefied oxygen and allowing to dispense liquefied oxygen to the container of liquefied oxygen;
a dispensing connection with a one-way check valve in communication with the container of liquefied inert gas and allowing to dispense liquefied oxygen to the container of liquefied inert gas;
a liquid-to-gas converter in communication with the container of liquefied oxygen and with the container of liquefied inert gas and receiving liquefied oxygen and liquefied inert gas separately from the container of liquefied oxygen and from the container of liquefied inert gas, evaporating them separately, and mixing evaporated oxygen and evaporated inert gas in a proportion so that to produce a mixture of a composition appropriate for breathing;
an inhalation unit in communication with the liquid-to-gas converter and releasing breathable gases around inhalation organs of a user;
a controlled valve disposed between outlet of the liquid-to-gas converter and the inhalation unit and opening and closing the flow of breathable gases from the liquid-to-gas converter to the inhalation unit;
a controller in communication with the controlled valve and transmitting activating signals to the controlled valve to open and close the flow of breathable gases;
a communicator in communication with the controller and receiving an input from a user and transmitting corresponding signal to the controller to open and to close the flow of breathable gases.

33. A breathing apparatus, comprising:
a container of liquefied oxygen storing and supplying a reasonable amount of liquefied oxygen;
a container of liquefied inert gas storing and supplying a reasonable amount of liquefied inert gas;
a dispensing connection with a one-way check valve in communication with the container of liquefied oxygen and allowing to dispense liquefied oxygen to the container of liquefied oxygen;
a dispensing connection with a one-way check valve in communication with the container of liquefied inert gas and allowing to dispense liquefied oxygen to the container of liquefied inert gas;
a liquid-to-gas converter in communication with the container of liquefied oxygen and with the container of liquefied inert gas and receiving liquefied oxygen and liquefied inert gas separately from the container of liquefied oxygen and from the container of liquefied inert gas, mixing them in a proportion so that to produce a mixture of a composition appropriate for breathing, and evaporating the mixture to produce gaseous breathable gases;
an inhalation unit in communication with the liquid-to-gas converter and releasing breathable gases around inhalation organs of a user;
a controlled valve disposed between outlet of the liquid-to-gas converter and the inhalation unit and opening and closing the flow of breathable gases from the liquid-to-gas converter to the inhalation unit;
a controller in communication with the controlled valve and transmitting activating signals to the controlled valve to open and close the flow of breathable gases;
a communicator in communication with the controller and receiving an input from a user and transmitting corresponding signal to the controller to open and to close the flow of breathable gases.

34. The apparatus of claims 30, 31, 32, and 33, wherein the dispensing connection being a quick-release connection.

35. The apparatus of claims 30, 31, 32, 33, and 34, wherein the elements of the apparatus are integrated to an automobile air conditioning system.

36. The apparatus of claims 30, 31, 32, 33, and 34, wherein the elements of the apparatus are integrated to a residential air conditioning system.

37. The apparatus of claim 36 comprising further a mobile platform, on which the containers of breathable gases are mounted so that to easily move them to a relevant dispenser of breathable gases.

38. The apparatus of claims 30, 31, 32, 33, and 34, wherein the elements of the apparatus are integrated to an automobile air conditioning system.

39. The apparatus of claims 30, 31, 32, 33, and 34, comprising further means for mounting the apparatus to a bicycle.

40. The apparatus of claims 30, 31, 32, 33, and 34, comprising further means for mounting the apparatus to a motorcycle.

41. A method of dispensing breathable gases to a container of compressed breathable gases, comprising:
delivering breathable gases to a dispenser of breathable gases;
connecting the container of breathable gases to the dispenser of breathable gases;
receiving an input from a user in a form of a payment transaction for dispensed breathable gases;
dispensing breathable gases in a paid amount to a container of breathable gases; and
disconnecting the container of breathable gases from the dispenser of breathable gases.

42. A method of dispensing liquefied breathable gases to a container of liquefied breathable gases, comprising:
delivering liquefied breathable gases to a dispenser of liquefied breathable gases;
connecting the container of liquefied breathable gases to the dispenser of liquefied breathable gases;
receiving an input from a user in a form of a payment transaction for dispensed liquefied breathable gases;
dispensing liquefied breathable gases in a paid amount to a container of liquefied breathable gases; and
disconnecting the container of liquefied breathable gases from the dispenser of liquefied breathable gases.

43. A method of dispensing liquefied breathable gases to a container of liquefied breathable gases, comprising:
delivering liquefied oxygen and liquefied inert gas to a dispenser of liquefied breathable gases;
mixing the liquefied oxygen and the liquefied inert gas in a proportion so that to produce a mixture of a composition appropriate for breathing;
connecting the container of liquefied breathable gases to the dispenser of liquefied breathable gases;
receiving an input from a user in a form of a payment transaction for dispensed liquefied breathable gases;
dispensing liquefied breathable gases in a paid amount to a container of liquefied breathable gases; and
disconnecting the container of liquefied breathable gases from the dispenser of liquefied breathable gases.

44. A method of dispensing liquefied breathable gases to a container of liquefied breathable gases, comprising:
delivering liquefied oxygen and liquefied inert gas to a dispenser of liquefied breathable gases;
connecting a container of liquefied oxygen and a container of liquefied inert gas to the dispenser of liquefied breathable gases;
receiving an input from a user in a form of a payment transaction for dispensed liquefied breathable gases;
dispensing liquefied oxygen and liquefied inert gas in a paid amount to the container of liquefied oxygen and to the container of liquefied inert gas respectively; and
disconnecting the container of liquefied oxygen and the container of liquefied inert gas from the dispenser of liquefied breathable gases.

45. A method of distributing breathable gases, comprising:
delivering breathable gases to a distributor of breathable gases;
receiving an input from a user in a form of a payment transaction for distributed breathable gases;
distribute breathable gases in a paid amount to one or more inhalation units; and
release breathable gases around inhalation organs of the user.

46. The method of claim 41 and claim 45, wherein the step of delivering breathable gases comprises:
receiving liquefied breathable gases from a source of liquefied breathable gases; and
evaporating the liquefied breathable gases to produce and deliver gaseous breathable gases to the dispenser of breathable gases.

47. The method of claim 41 and claim 45, wherein the step of delivering breathable gases comprises:
receiving liquefied oxygen and liquefied inert gas separately from a source of liquefied breathable gases;
mixing the liquefied oxygen and the liquefied inert gas in a proportion to produce a mixture of a composition appropriate for breathing;
evaporating the mixture of liquefied oxygen and liquefied inert gas to produce and deliver gaseous breathable gases to the dispenser of breathable gases.

48. The method of claim 41 and claim 45, wherein the step of delivering breathable gases comprises:
receiving liquefied oxygen and liquefied inert gas separately from a source of liquefied breathable gases;
evaporating the liquefied oxygen and the liquefied inert gas separately;
mixing evaporated oxygen and evaporated inert gas in a proportion to produce a mixture of a composition appropriate for breathing.
